(19) United States
(12) Patent Application Publication
(10) Pub. No.: US 2022/0169689 A1
(43) Pub. Date: Jun. 2, 2022

(54) STAT3 TRANSCRIPTOME FOR DESIGNING MORE POTENT NK CELLS

(71) Applicant: RESEARCH INSTITUTE AT NATIONWIDE CHILDREN'S HOSPITAL, Columbus, OH (US)

(72) Inventors: Nitin CHAKRAVARTI, Philadelphia, PA (US); Dean Anthony LEE, Canal Winchester, OH (US)

(21) Appl. No.: 17/437,400

(22) PCT Filed: Mar. 9, 2020
(86) PCT No.: PCT/US2020/021737

§ 371 Date: Sep. 8, 2021

Related U.S. Application Data
(60) Provisional application No. 62/815,625, filed on Mar. 8, 2019.

(51) Int. Cl. C07K 14/47; C12N 5/0783; A61K 35/17

(52) U.S. Cl. CPC C07K 14/4705; C12N 5/0646; C12N 2501/2315; C12N 2501/2321; A61K 35/17

(57) ABSTRACT

Disclosed are expanded NK cell compositions comprising, in some aspects, activated STAT3 transcriptomes and methods of using the same to treat, inhibit, reduce, ameliorate, and/or prevent diseases.

This application claims the benefit of U.S. Provisional Application No. 62/815,625, filed on Mar. 8, 2019, which is incorporated herein by reference in its entirety.

### I. BACKGROUND

Immunotherapy is the treatment of disease by activating or suppressing the immune system. Cells derived from the immune system may be manipulated and modified as cell based therapies intended to improve immune functionality and characteristics. In recent years, immunotherapy has become of great interest to researchers, clinicians and pharmaceutical companies, particularly in its promise to treat various forms of cancer. Immunomodulatory regimens often have fewer side effects than existing drugs, including less potential for creating resistance when treating infectious diseases (microbial and viral) disease. Cell based therapies such as CAR-T approaches have various barriers such as the non-availability of a targetable antigen, costs involved for production and delivery and side effects.

Conventional cancer treatments focus on killing or removing cancer cells with chemotherapy, surgery, and/or radiation. However, the field of therapeutic immune cells is growing rapidly, and can be used in conjunction with or, in some cases, in place of conventional treatments to treat, prevent, or delay the onset of a cancer. Immune effector cells such as lymphocytes, macrophages, dendritic cells, natural killer cells (NK Cell), cytotoxic T lymphocytes (CTL), etc., naturally work together to defend the body against cancer by targeting abnormal antigens expressed on the surface of tumor cells. Natural killer (NK) cells are often the first line of defense against aberrant cells resulting from viral infection or malignant transformation, and restoration of NK cell function by adoptive transfer or potential in vivo stimulation is a promising therapy for cancer or other maladies made possible by ex vivo cultivation to increase NK cell number and improve effector function. What are needed are new improved way to expand and activate NK cells for use in these therapies.

### II. SUMMARY

Disclosed are methods and compositions for expanding and modifying natural killer cells, wherein the modified cells optionally comprise a modified, non-naturally occurring STAT3 transcriptome, as described in more detail below

In one aspect, disclosed herein are expanded, and modified natural killer cells comprising a non-naturally occurring, consisting of state having a modified STAT3 transcriptome, through the use of an engineered membrane bound IL-21 (mbIL-21) in various forms. In one aspect, modified natural killer cells can be attained by artificially manipulating the structural state of the genome or gene expression systems resulting in a modified natural killer cell having altered epigenetics, altered transcriptomics, and/or altered ability to respond to external stimuli and enhanced phenotype, when compared to a naïve natural killer cell. In one aspect, the expanded modified natural killer cells may comprise a Modified STAT3 transcriptome that may include one or more differentially expressed genes involved in telomere organization, regulation of mitosis, DNA repair, immunity, cytokine signaling, altered metabolism, glycolysis, gluconeogenesis, cytotoxicity activation, p53 pathway (such as, for example, any of those genes disclosed in FIG. 3 or 4, including, but not limited to HIST1H1B, HIST1H2AB, HIST1H2AG, HIST1H2AH, HIST1H2AI, HIST1H2AJ, HIST1H2AL, HIST1H2BB, HIST1H2BE, HIST1H2BH, HIST1H2BJ, HIST1H2BL, HIST1H2BM, HIST1H2BO, HIST1H3B, HIST1H3C, HIST1H3F, HIST1H3G, HIST1H3H, HIST1H3J, HIST1H4A, HIST1H4D, HIST1H4L, HIST2H3C, HIST2H4B, HIST3H2BA, HIST3H2BB, ABCA1, AK5, ASCL2, B3GAT1, CACNA2D2, CCR5, CXCR2, ENPP5, FBLN2, FCRL3, GNAL, GPRASP1, GRIK4, GTSE1, HIST3H2BB, IL1B, ITGA2, KIF13A, KIF4A, LINC00599, LONRF3, LRRN3, MSC-AS1, NFIX, NUAK1, NUAK2, PCDH1, PLXNA4, RAD51, RNF157, SLC1A7, SPON2, TYMS, WWC2, ZNF442, ZNF727, and ZSCAN18).

Also disclosed herein are natural killer cells of any preceding aspect, wherein the ratio of down-regulated to overexpressed genes or up-regulated to suppressed gene is about 1.5.

In one aspect, disclosed herein are modified natural killer cells of any preceding aspect, wherein the natural killer cell comprises an upregulated one or more protein selected from the group consisting of BIRCS, MK167, TOP2A, CKS2 and RACGAP1.

Also disclosed herein are modified natural killer cells of any preceding aspect, wherein the natural killer cell comprises a downregulated one or more protein selected from the group consisting of PTCH1, TGFB3, and ATM.

The present disclosure also encompasses expanded, modified natural killer cells of any preceding aspect, wherein the cells are expanded, modified in vivo or ex vivo by contacting the NK cell with IL-21, IL-15, and/or 4-BBL. In one aspect, the IL-21, IL-15, and/or 4-BBL are provided on the surface of feeder cells, plasma membrane vesicles, liposomes, and/or exosomes. Thus, in one aspect, disclosed herein are natural killer cells of any preceding aspect, wherein the cells are expanded, modified in vivo or ex vivo by contacting the NK cell with a plasma membrane vesicle, liposome, exosome, or feeder cell that was engineered to express membrane bound IL-21, IL-15, and/or 4-BBL. In one aspect, the contact with the IL-21, IL-15, and/or 4-BBL can occur for 5, 6, 7, 8, 9, 10, 15, 20, 25, 30, 35, 40, 45, 50, 55, 60, 65, 70, 75, 80, 85, 90, 95, 100, 105, 110, 115, 120, 150 minutes, 3, 4, 5, 6 7, 8, 9, 10, 11, 12, 13, 14, 15, 16, 17, 18, 19, 20, 21, 22, 23, 24, 30, 32, 36, 42, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30, 31, 45, 60, 61, 62 days, 3, 4, 5, or 6 months.

Also disclosed herein is a method of treating, inhibiting, reducing, ameliorating, and/or preventing a cancer and/or metastasis in a subject comprising administering to the subject a therapeutically effective amount of the expanded, modified natural killer cell of any preceding aspect.

In one aspect, disclosed herein is a method of modulating (i.e., increasing or decreasing) the immune system (for example an immune response) of a subject, comprising administering an effective amount of expanded, modified natural killer cells comprising an activated modified STAT3 transcriptome, or administering an agent to activate endogenous NK cells by activating the Modified STAT3 transcriptome and thereby obtaining modified NK cells in vivo

### III. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, 1C, 1D, 1E, and 1F show genome-wide STAT3-binding in naïve and ex vivo expanded, modified NK cells. FIG. 1A shows Western blot showing phosphorylation of STAT3 in response to IL-21 stimulation. NK cells were treated with IL-21 for 30 minutes and protein was extracted and blotted for total STAT3, phospho-Y705-STAT3 (pSTAT3), and β-actin as a loading control. FIG. 1B shows Venn diagrams displaying STAT3 ChIP-seq peak summit (as defined by MACS) across the stimulated-naive (red) and -expanded, genetically modified (blue) NK cells. FIG. 1C shows that STAT3 binds to its own promoter in IL-21-treated NK cells. This is a genomic view of the STAT3 ChIP-seq tag density around the STAT3 gene. FIG. 1D shows de novo motifs recovered by HOMER from the entire list of STAT3 binding sites (right motif) in naïve and expanded, genetically modified NK cells. FIG. 1E shows spatial distribution and Heatmap representation of the distance of STAT3 binding around the TSS of all protein-coding genes in NK cells treated with IL-21 for 30 min. FIG. 1F shows distribution of STAT3 binding site location relative to RefSeq genes. Locations of binding sites are divided into distal promoter (−1 to −3 kb upstream of TSS), proximal promoter (−1 to O kb), 5′ UTR, exon, intron, 3′ UTR, proximal downstream (0-1 kb downstream of TSS), distal downstream (1 to 3 kb downstream ofTSS) and distal intergenic (>3 kb from genes).

FIGS. 2A and 2B shows gene Ontology (GO) analysis of genes identified by STAT3 ChIP in (2A) naive and (2B) expanded, genetically modified NK cells. Over-represented GO terms resulting from the analysis of all in STAT3-binding sites with GREAT, showing Biological Process (green), the Molecular Function (crimson), and the PANTHER signaling pathways (blue).

FIGS. 3A, 3B, 3C, and 3D show whole-transcriptome analysis of IL-21 stimulated naive and expanded modified NK cells. FIG. 3A shows volcano plots representing upregulated and downregulated transcripts of expanded & naive NK cells in response to IL-21 stimulation. The logarithms of the fold changes of individual genes (x-axis) are plotted against the negative logarithm of their p-value to base 10 (y-axis). Positive log 2 (fold change) values represent upregulation in expanded NK cells compared to naive cells, and negative values represent downregulation. Circles above the dotted line represent differentially expressed genes between asthma and controls with p<0.05 after correction for multiple testing. FIG. 3B shows scatter plot showing the expression values of all assembled transcript fragments. Expression is shown as the log 2 of the FPKM, including up-regulated transcripts (green) and down-regulated transcripts (red). FIG. 3C shows a heat map of the top 100 enriched and depleted transcripts across different donors. Color-coding is based on dog-transformed read count values. FIG. 3D shows the identification of GO pathways in stimulated NK cells.

FIGS. 4A and 4B show the ingenuity Pathway Analysis (IPA)-based network associated with NK cell functions. FIG. 4A shows IPA-based pathway analysis of the list of genes that are differentially expressed in IL-21 stimulated, expanded, and modified and naïve NK cells (P-value:′.S 0.01 and fold-change 2: 1.5). FIG. 4B shows the top scoring regulatory networks identified with the IPA software corresponded to Cell Death and Survival, Infectious Diseases, Cellular Function and Maintenance, and Hematological Disease. Genes that are upregulated and downregulated in ex vivo expanded, activated and thus modified NK cells (when compared with the naive ones) are displayed within red and green nodes, respectively. Solid and dashed lines between genes represent known direct and indirect gene interactions, respectively. The shapes of the nodes reflect the functional class of each gene product: transcriptional regulator (horizontal ellipse), transmembrane receptor (vertical ellipse), enzyme (vertical rhombus), cytokine/growth

FIGS. 5A and 5B show that STAT3 binding regulates the expression of nearby genes. Summary of selected categories of over-represented GO terms as calculated by Enrichr. (5A) Up-regulated genes and (5B) Down-regulated genes, were used to determine GO term over-representation. The GO categories are listed on the left and ranked by their associated log IO p-values.

FIG. 6 shows the activation of the STAT3-mediated pathways in expanded, modified NK cells. Functional network analysis by IPA using RNA-seq data is shown for the cells at 4 hours in the IL-21 stimulated state. The genes involved in the top scoring pathway (i.e. IFNγ signaling) and their interaction with the STAT3 pathway are represented. Nodes in red indicate up-regulation in the cases and nodes in green indicate downregulation (actor (square), kinase (inverted triangle) and complex/group/other (circle)).

FIG. 7 shows a plot of the ATACseq showing a direct correlation with opening of the chromatin and gene expression

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

As various changes could be made in the above-described cells and methods without departing from the scope of the invention, it is intended that all matter contained in the above description and in the examples given below, shall be interpreted as illustrative and not in a limiting sense.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of a disease or an infection.

The term “NK cell(s)” is an abbreviation for “natural killer cell(s)”, and the two terms are used interchangeably herein.

The term “modified” as used herein to describe NK cells, refers to cells having an artificially altered epigenome, transcriptome, and/or an artificially altered proteome such that the epigenome, transcriptome and/or proteome are artificial. Any one or all of an altered epigenome, transcriptome or proteome can result in artificially altered cell function, as compared to a naïve NK cell not subjected to the methods described herein or contacted with the engineered compositions described herein. A modified NK cell may be an activated NK cell relative to a naïve NK cell, or may be an NK cell with other beneficial characteristics, compared to the naïve NK cell. An “activated” NK cell is a cell with an artificially induced phenotype reflecting increased NK cell function, such as increased cytotoxicity, increased longevity or viability, increased physiological persistence, altered ability to respond to external stimuli, increased metabolism or the like. A modified NK cell as disclosed herein is not necessarily an activated NK cell, but an activated NK cell is a modified NK cell.

As used herein, the term “genetically engineered” describes natural killer cells in which a gene may be further modified using by a specific change of genetic sequence, such as a point mutation, gene insertion, gene deletion, transposition, or any change in the sequence of DNA base pairs. The change in DNA base pair sequence may be introduced through viral vectors such as retroviral methods, lentiviral methods, adenoviral (AAV) methods, Cre-lox methods, meganuclease methods, TALEN methods, CRISPR-based (e.g., CRISPR-Cas9) methods, transposase methods, chemical alteration, or any other DNA base pair sequence editing methods.

As used herein, the term “differentially expressed” describes a characteristic of STAT3-modulated genes identified with activation of the STAT3 transcriptome, meaning that expression of a gene is either increased (i.e., up-regulated) or decreased (i.e, down-regulated) by at least 50% relative to a naïve NK cell (i.e., a fold change in expression ratio ≥1.5).

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular modified natural killer (NK) cell is disclosed and discussed and a number of modifications that can be made to a number of molecules including the modified NK cell are discussed, specifically contemplated is each and every combination and permutation of a modified NK cell and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

In one aspect, disclosed herein are modified natural killer cells comprising an activated STAT3 transcriptome.

Natural Killer Cells are a type of cytotoxic lymphocyte of the immune system. NK cells provide rapid responses to virally infected cells and respond to transformed cells. In contrast to NK cells, T cells detect peptides from pathogens presented by Major Histocompatibility Complex (MHC) molecules on the surface of infected cells, triggering cytokine release, causing lysis or apoptosis. NK cells are unique, however, as they have the ability to recognize stressed cells regardless of whether peptides from pathogens are present on MHC molecules. They were named “natural killers” because of the initial notion that they do not require prior activation in order to kill target. NK cells are large granular lymphocytes (LGL) and are known to differentiate and mature in the bone marrow from where they then enter into the circulation. In some aspect, the NK cell can be a genetically engineered CAR NK cell or may include other gene insertions or deletions achieved for example using a chemical or viral vector, or a vector delivery system. Optionally, targeted genome editing techniques may be used, such as meganucleases, zinc finger nucleases, transcription activator-like effector nucleases (TALENs), and CRISPR-based using a guide RNA (gRNA).

Because it is helpful to be able to administer large numbers of immune cells (such as, for example, NK cells including, but not limited to memory-like NK cells, CAR NK cells, and activated NK cells) during immunotherapy, in some embodiments the modified immune cells are expanded immune cells. Expanded immune cells are those that are grown ex-vivo starting from an initial population of cells, in order to obtain a large number of immune cells. In some embodiments, the expanded immune cells are autologous cells that yet can be easily administered to a subject without provoking an immune response. However, in some embodiments, the expanded immune cells are allogeneic immune cells, in which their inherent alloreactivity can be a benefit. In further embodiments, the expanded immune cells are further genetically engineered to include chimeric antigen receptors to help the immune cells target diseased tissue. Genetically engineered natural killer cells may be produced through the engineered change of the genetic sequence, such as a gene insertion, gene deletion, transposition, or any change in the sequence DNA base pairs. The change in DNA base pair sequence may be introduced through viral vectors such as retroviral methods, lentiviral methods, adenoviral (AAV) methods, Cre-lox methods, meganuclease targeting methods, zinc finger nuclease targeting methods, CRISPR-based (e.g. CRISPR-Cas9targeting methods, transposase methods, chemical alteration, or any other DNA base pair sequence editing methods. Preparation of expanded immune cells includes both activating and expanding the immune cells. A number of cytokines (IL-2, IL-12, IL-15, IL-18, IL-21, type I IFNs, and TGF-β) have been shown to be useful for modifying and expanding immune cells ex vivo. For example, in some embodiments, the NK cells being evaluated are IL-21 expanded NK cells. Accordingly, in one aspect, disclosed herein are immunotherapy methods further comprising expanding the at least one potent immune cell prior to delivering a therapeutically effective amount of the potent immune cell.

As noted above, the current disclosure relates to expanded, modified natural killer cells. Expansion refers broadly to the ex vivo proliferation of NK cells so that the population of NK cells is increased. Activation refers to the stimulation of NK cells through various artificial means and methods as described herein, to manipulate the epigenetic, transcriptomic, phenotypic state of the NK cells. Altered phenotypic state may include any one or more of the following modifications, relative to a naïve NK cell: altered gene regulation (transcriptional and translational), altered imprinting through epigenetics, receptor phenotype expression, altered metabolism, altered cytotoxicity toward targets, altered memory-like phenotypes, and the like. The induced transcriptional characteristics within an NK cells stimulated by IL-21 bearing feeder cells, PM-particles, or exosomes can be manifested through proteomic characteristics and furthermore bear out in altered unique functional phenotype for metabolism, cell killing activity, and other helpful function. Modified NK cells as described herein can be expanded and activated, for example, from peripheral blood mononuclear cells. However, modified NK cells can also be prepared from other types of cells, such as hematopoietic stem cells or progenitor cells. The initial blood or stem cells can be isolated from a variety of different sources, such placenta, umbilical cord blood, placental blood, peripheral blood, spleen or liver. Other sources can include NK cells differentiated from iPSCs or ESCs. Preparation occurs in a cell culture medium. Suitable cell culture mediums are known to those skilled in the art. The modified NK cells can be a provided as a cell line, which is a plurality of cells that can be maintained in cell culture. Modified NK cells may be imprinted through changes in epigenetic patterns, by IL-21 stimulations, such that a phenotype may be transmitted to daughter cells upon expansion. Such modified NK cells could be cryopreserved, then potentially again further modified after thaw. Thus, in one aspect, disclosed herein are immunotherapy methods further comprising modifying the at least one potent immune cell prior to delivering a therapeutically effective amount of the potent immune cell. In some aspects, the immune cell has been extracted from a subject using known methods prior to performing the method of determining the potency of the immune cell. Alternatively, the immune cell can be sourced from expansion of a cell culture.

As noted throughout this application, the natural killer cells disclosed herein are structurally modified, meaning they are not only expanded, but also have a structurally altered, artificial transcriptome and/or proteome, and may be activated. In one aspect disclosed herein are modified NK cells, wherein the modified NK cells are modified in vivo or ex vivo by contacting a naïve or previously treated NK cell with IL-21, IL-15, and/or 4-BBL. In one aspect, the IL-21, IL-15, and/or 4-BBL are provided on the surface of one or more feeder cells, plasma membrane vesicles, liposomes and/or exosomes, or any combination thereof. Thus, in one aspect, disclosed herein are modified NK cells, wherein the modified NK cells are modified in vivo or ex vivo by contacting the NK cells with a plasma membrane vesicle, liposome, exosome, or feeder cell or any combination thereof which is engineered to express membrane bound IL-21, IL-15, and/or 4-BBL.

Plasma membrane (PM) particles are vesicles made from the plasma membrane of a cell or artificially made (i.e., liposomes). A PM particle can contain a lipid bilayer or simply a single layer of lipids. A PM particle can be prepared in single lamellar, multi-lamellar, or inverted form. PM particles can be prepared from mbIL21-bound feeder cells as described herein, using known plasma membrane preparation protocols or protocols for preparing liposomes such as those described in U.S. Pat. No. 9,623,082, the entire disclosure of which is herein incorporated by reference. In certain aspects, PM particles as disclosed herein range in average diameter from about 170 to about 300 nm.

Exosomes are cell-derived vesicles that are present in many and perhaps all eukaryotic fluids. Exosomes contain RNA, proteins, lipids and metabolites that is reflective of the cell type of origin. The reported diameter of exosomes is between 30 and 100 nm. Exosomes are either released from the cell when multivesicular bodies fuse with the plasma membrane or released directly from the plasma membrane. In some embodiments, exosomes are obtained from cancer cells. In some embodiments, the exosomes are leukemic cell exosomes. While this disclosure is given in the context of using exosomes to determine the potency of an immune cell, other extracellular vesicles may also be used to determine the potency of an immune cell. As used herein, the term “extracellular vesicle” includes, but is not limited to, all vesicles released from cells by any mechanism. “Extracellular vesicles” includes exosomes which are released from multivesicular bodies and microvesicles that are shed from the cell surface. “Extracellular vesicles” includes vesicles created by exocytosis or ectocytosis. “Extracellular vesicles” encompasses exosomes released from multivesicular bodies, vesicles released by reverse budding, fission of membrane(s), multivesicular endosomes, ectosomes, microvesicles, microparticles, and vesicles released by apoptotic bodies, and hybrid vesicles containing plasma membrane components. Extracellular vesicles can contain proteins, nucleic acids, lipids, and other molecules common to the originating cell.

In one aspect, the plasma membrane particles, feeder cells, liposomes, exosomes or any combination thereof can be purified from feeder cells that stimulate immune cells (such as, for example NK cells). Immune cell stimulating feeder cells for use in the claimed invention, for use in making the engineered plasma membrane particles, engineered feeder cells, engineered liposomes, or engineered exosomes disclosed herein can be either irradiated autologous or allogeneic peripheral blood mononuclear cells (PBMCs) or nonirradiated autologous or allogeneic PBMCs, RPMI8866, HFWT, 721.221, K562 cells, EBV-LCLs, T cells transfected with one or more membrane bound IL-21, membrane bound IL-15, membrane bound 4-1BBL, membrane bound OX40L and/or membrane TNF-α, (such as for example, T cells transfected with membrane bound IL-21, T cells transfected with membrane bound 4-1BBL, T cells transfected with membrane bound IL-15 and 4-1BBL, T cells transfected with membrane bound IL-21 and 4-1BBL), NK cells (including, but not limited to PBMCs, RPM18866, NK-92, NK-92MI, NK-YTS, NK, NKL, KIL, KIL C.2, NK 3.3, NK-YS, HFWT, K562 cells, autologous cancer cells) transfected with membrane bound IL-21, NK cells (including, but not limited to PBMCs, RPMI8866, NK-92, NK-92MI, NK-YTS, NK, NKL, KIL, KIL C.2, NK 3.3, NK-YS, HFWT, K562 cells, autologous cancer cells) transfected with membrane bound 4-1BBL, NK cells (including, but not limited to PBMCs, RPMI8866, NK-92, NK-92MI, NK-YTS, NK, NKL, KIL, KIL C.2, NK 3.3, NK-YS, HFWT, K562 cells, autologous cancer cells) transfected with membrane bound IL-15 and 4-1BBL, or NK cells (including, but not limited to PBMCs, RPMI8866, NK-92, NK-92MI, NK-YTS, NK, NKL, KIL, KIL C.2, NK 3.3, NK-YS, HFWT, K562 cells, autologous cancer cells) transfected with membrane bound IL-21 and 4-1BBL as well as other non-HLA or low-HLA expressing cell lines or patient derived primary tumors.

The plasma membrane particle, feeder cells, liposomes, exosomes and/or any combination thereof used in the disclosed methods or to modify the disclosed modified NK cells, can further comprise additional effector agents to modify, including expanding and/or activating immune cells (such as, for example, NK cells). Thus, in one aspect disclosed herein are modified NK cells, wherein the feeder cells used to generate the disclosed engineered liposomes, engineered exosomes, engineered feeder cells, engineered plasma membrane particles or any combination thereof, further comprise at least one additional immune cell effector agent on its cell surface, wherein the at least one additional immune cell effector agent is a cytokine, an adhesion molecule, or an immune cell activating agent (such as, for example, 4-1BBL, IL-2, IL-12, IL-15, IL-18, IL-21, MICA, LFA-1, 2B4, CCR7, OX40L, UBLP2, BCM1/SLAMF2, NKG2D agonists, CD137L, CD137L, CD155, CD112, Jagged1, Jagged2, Delta-1, Pref-1, DNER, Jedi, SOM-11, wingless, CCN3, MAGP2, MAGP1, TSP2, YB-1, EGFL7, CCR7, DAP12, and DAP10, Notch ligands, NKp46 agonists, NKp44 agonists, NKp30 agonists, other NCR agonists, CD16 agonists). In one aspect the at least one additional immune cell effector agent comprises IL-21, 4-1BBL, IL-15, IL-21 and 4-1BBL, IL-21 and IL-15, or IL-15 and 4-1BBL. Accordingly, in one aspect, the feeder cells, liposomes, plasma membrane particles, exosomes or any combination thereof, generated by said feeder cells can comprise membrane bound versions of any combination of the immune cell activating agents (such as, for example, 4-1BBL, IL-2, IL-12, IL-15, IL-18, IL-21, MICA, LFA-1, 2B4, CCR7, OX40L, UBLP2, BCM1/SLAMF2, NKG2D agonists, CD137L, CD155, CD112, Jagged1, Jagged2, Delta-1, Pref-1, DNER, Jedi, SOM-11, wingless, CCN3, MAGP2, MAGP1, TSP2, YB-1, EGFL7, CCR7, DAP12, and DAP10, Notch ligands, NKp46 agonists, NKp44 agonists, NKp30 agonists, other NCR agonists, CD16 agonists). For example, the exosomes or plasma membrane particles can have IL-15, IL-21, and/or 4-1BBL on their membrane. In one aspect, the NK cells can be expanded with soluble 4-1BBL, IL-2, IL-12, IL-15, IL-18, IL-21, MICA, LFA-1, 2B4, CCR7, OX40L, UBLP2, BCM1/SLAMF2, NKG2D agonists, CD137L, CD155, CD112, Jagged1, Jagged2, Delta-1, Pref-1, DNER, Jedi, SOM-11, wingless, CCN3, MAGP2, MAGP1, TSP2, YB-1, EGFL7, CCR7, DAP12, and DAP10, Notch ligands, NKp46 agonists, NKp44 agonists, NKp30 agonists, other NCR agonists, CD16 agonists that can be added directly to an ex vivo culture, administered to a subject receiving the NK cells, or secreted by feeder cells, plasma membrane vesicles, liposomes, or exosomes in culture ex vivo or in vivo. Thus, it is understood and herein contemplated that the NK cells can be expanded ex vivo or in vivo.

It is understood and herein contemplated that the immune cells must be exposed to the particle or exosome for a period of time sufficient to be induced to produce cytokines. In one aspect, disclosed herein are methods of assaying the potency of an immune cell wherein the immune cell is contacted with an effective amount of a plasma membrane particle, a liposome, an exosome or any combination thereof, (including, but not limited to engineered particles, liposomes, and/or exosomes) for at least 5, 6, 7, 8, 9, 10, 15, 20, 25, 30, 35, 40, 45, 50, 55, 60, 65, 70, 75, 80, 85, 90, 95, 100, 105, 110, 115, 120, 150 minutes, 3, 4, 5, 6 7, 8, 9, 10, 11, 12, 13, 14, 15, 16, 17, 18, 19, 20, 21, 22, 23, 24, 30, 32, 36, 42, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30, 31, 45, 60, 61, 62 days, 3, 4, 5, or 6 months.

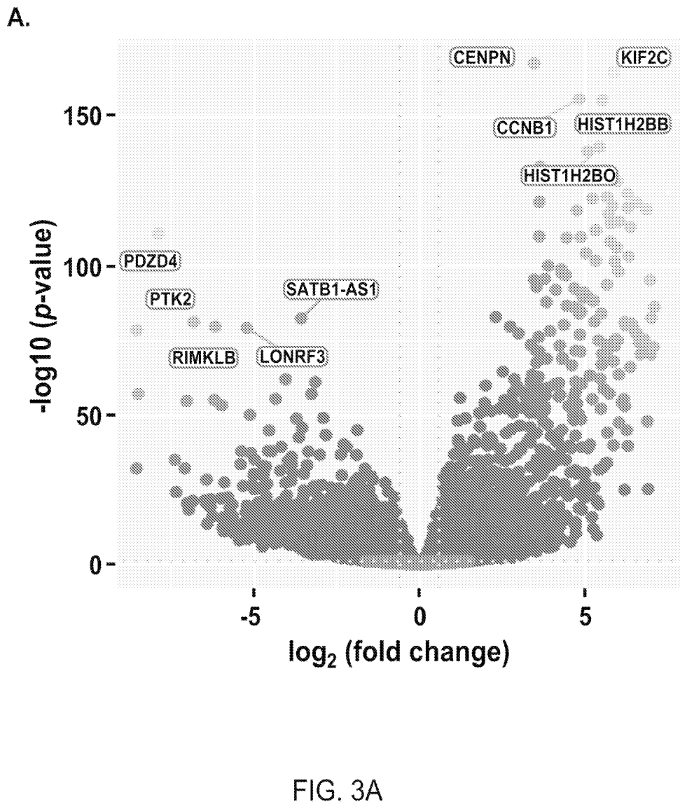

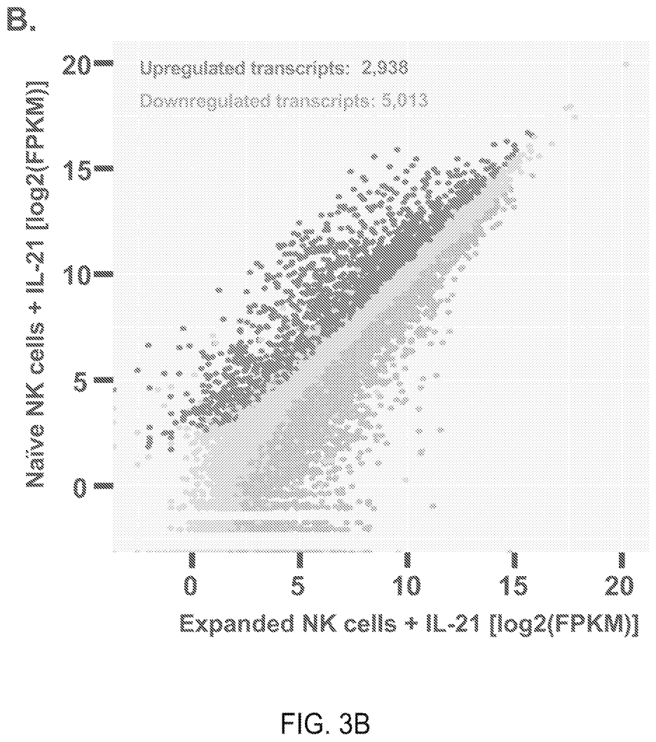

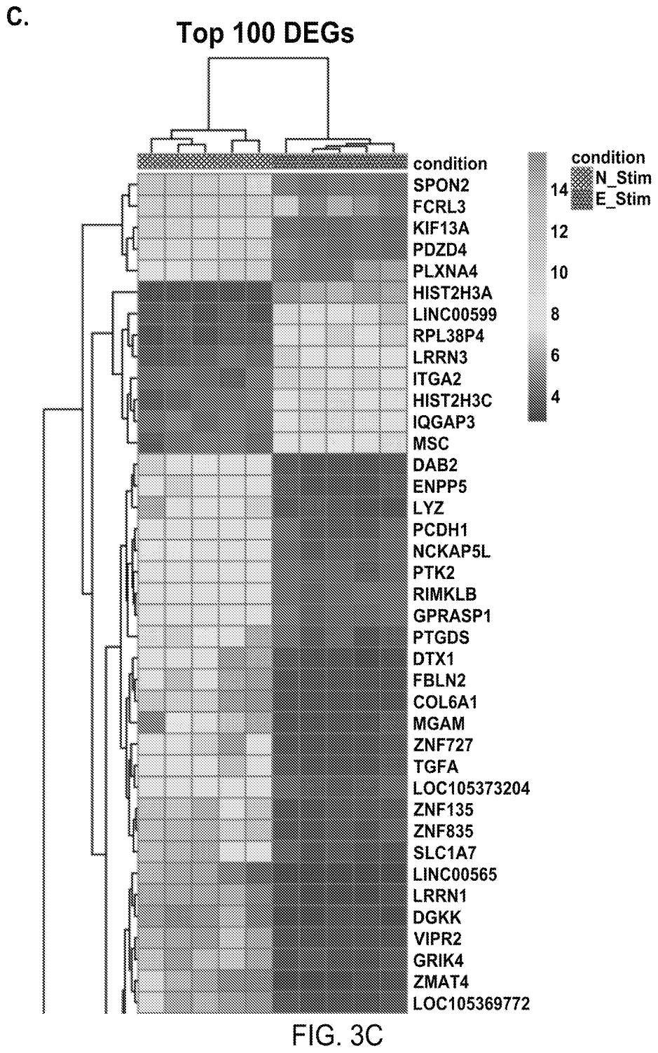

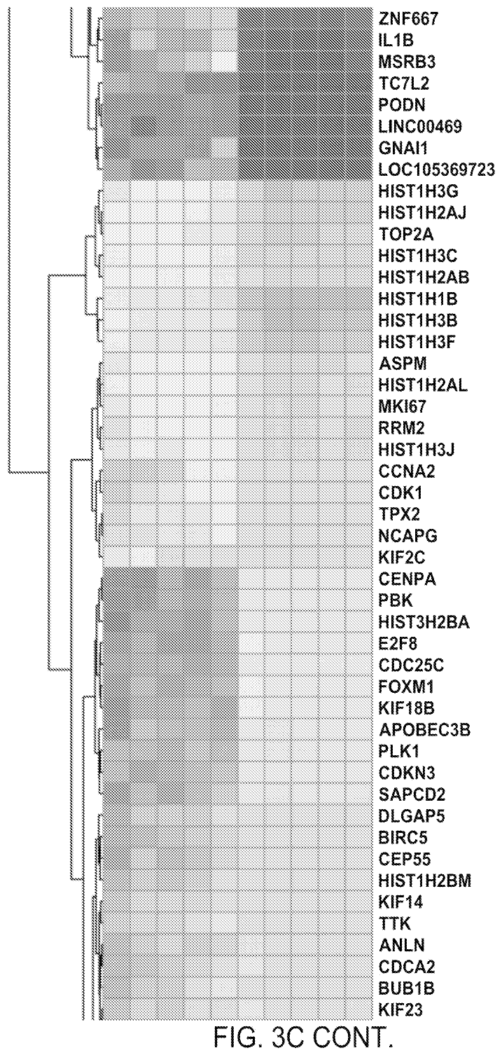

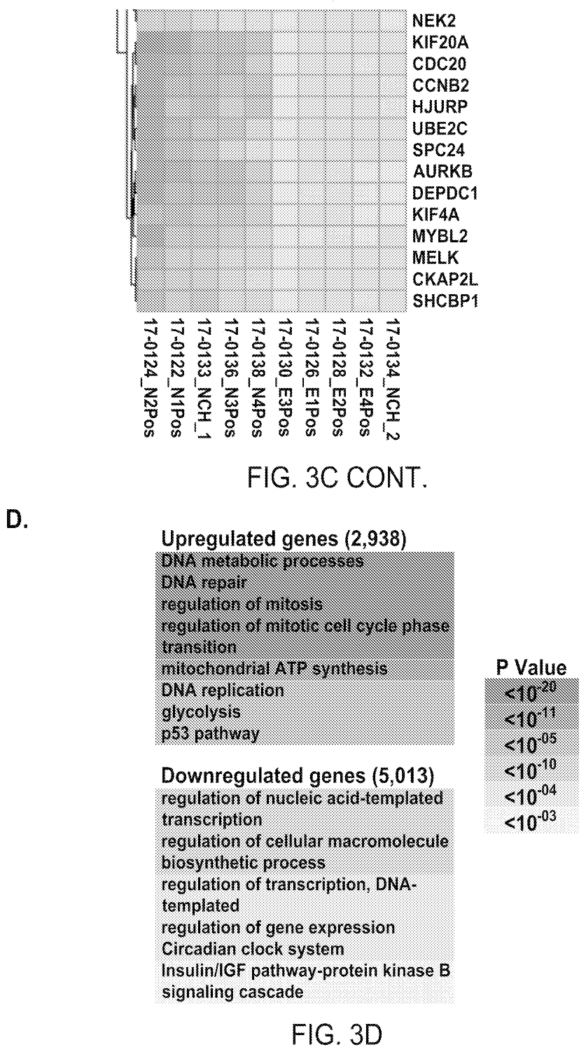

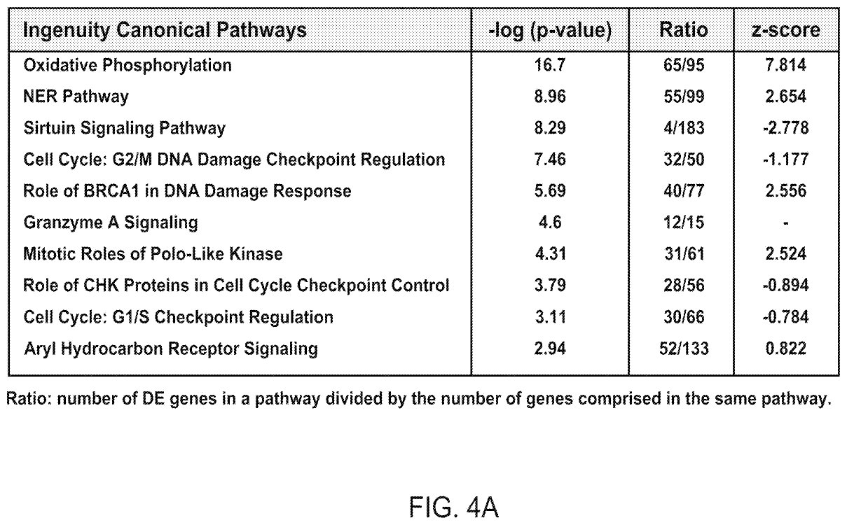

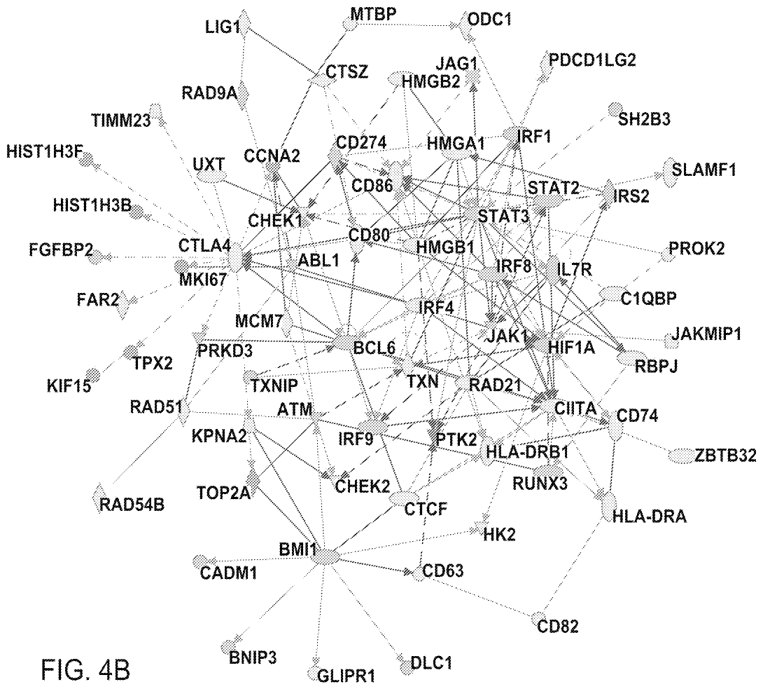

In one aspect, the Modified STAT3 transcriptome of the modified NK cell can comprise one or more differentially expressed genes involved in telomere organization, regulation of mitosis, DNA repair, immunity, cytokine signaling, glycolysis, gluconeogenesis, p53 pathway (such as, for example, any of those genes disclosed in FIG. 3 or 4, including, but not limited to HIST1H1B, HISTIH2AB, HISTIH2AG, HISTIH2AH, HISTIH2AI, HISTIH2AJ, HISTIH2AL, HISTIH2BB, HISTIH2BE, HISTIH2BH, HISTIH2BJ, HISTIH2BL, HISTIH2BM, HIST1H2BO, HISTIH3B, HISTIH3C, HISTIH3F, HISTIH3G, HISTIH3H, HISTIH3J, HISTIH4A, HISTIH4D, HISTIH4L, HIST2H3C, HIST2H4B, HIST3H2BA, HIST3H2BB, ABCA1, AK5, ASCL2, B3GAT1, CACNA2D2, CCR5, CXCR2, ENPP5, FBLN2, FCRL3, GNAL, GPRASP1, GRIK4, GTSE1, HIST3H2BB, IL1B, ITGA2, KIF13A, KIF4A, LINC00599, LONRF3, LRRN3, MSC-AS1, NFIX, NUAK1, NUAK2, PCDH1, PLXNA4, RAD51, RNF157, SLC1A7, SPON2, TYMS, WWC2, ZNF442, ZNF727, and ZSCANi8).

In one aspect, disclosed herein are any of the modified NK cells disclosed herein, wherein the ratio of down-regulated to overexpressed genes is about 1.5. For example, any of the modified NK cells disclosed herein, may comprise an upregulated one or more protein selected from the group consisting of BIRCS, MK167, TOP2A, CKS2 and RACGAP1. Also disclosed herein are modified NK cells, wherein the natural killer cell comprises a downregulated one or more protein selected from the group consisting of PTCH1, TGFB3, and ATM.

1. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

C. Method of Treating Cancer

It is understood and herein contemplated that the modified NK cells, for example those comprising an activated Modified STAT3 transcriptome as disclosed herein, can modulate immune response. Thus, in one aspect, disclosed herein are methods of modulating the immune system of a subject, comprising administering an effective amount of modified NK cells comprising an activated STAT3 transcriptome. Alternatively, the present disclosure contemplates administering an agent, such as a PM particle, exosome, or any combination thereof to a subject in need thereof, to modulate the immune system of the subject by stimulating endogenous NK cells to a state of NK cells with an activated STAT3 transcriptome.

In one aspect, it is understood that modulating immune responses can have a clinically beneficial effect on disease states and thus serve as a therapeutic for a disease or condition or augment another therapy used to treat a disease or condition. The disclosed compositions can be used to treat any disease where uncontrolled cellular proliferation occurs such as cancers. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer. Thus, in one aspect, disclosed herein are methods of treating, inhibiting, reducing, ameliorating, and/or preventing a cancer and/or metastasis in a subject comprising administering to the subject a therapeutically effective amount of the expanded natural killer cell as disclosed herein.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: The Altered STAT3 Transcriptome in NK Cells During Proliferative Expansion Leads to Reprogramming of Metabolic, Epigenetic, and Survival Networks

Despite their discovery more than four decades ago, natural killer (NK) cells have only recently gained attention for their potential in the cellular therapy. NK cells are an important part of the innate immune system and play a critical role in host immunity against microbial infection and tumor progression. A growing number of clinical studies have underlined the promising anti-tumor effects of NK cell-based immunotherapy. However, insufficient number and limited lifespan are obstacles that limit the application of NK cells in adaptive immunotherapy. Multiple approaches are being used to enhance the number and function of NK cells, including cytokines. Earlier, we successfully demonstrated robust NK-cell expansion following co-culture with feeder cells expressing membrane-bound interleukin-21 (mbIL21), without undergoing senescence up to 6 weeks in culture. However, the transcriptional impact of IL-21 signaling has not been fully evaluated.

IL-21 is a type I cytokine essential for NK-cell activation, maturation and proliferation. However, the molecular mechanisms underlying effects of IL-21 in NK cells are not yet completely understood. IL-21 signals via the Janus kinase (JAK) and signal transducer and activator of transcription (STAT) pathway. Binding of IL-21 to the IL-21 receptor results in the phosphorylation of JAK and concurrent activation of STAT3. Upon phosphorylation, STAT3 forms a homodimer which translocate to the nucleus and initiates transcription of the IL-21 responsive genes.

STAT3 is a negative regulator of inflammation and inhibiting STAT3 in tumors has been shown to enhance anti-tumor immunity. In contrast, STAT3 is an integral part of the signal transduction cascade in human NK cell development and has multiple functions involving apoptosis, survival and proliferation. STAT3 is involved in driving almost all of the pathways that control NK cytolytic activity as well as the reciprocal regulatory interactions between NK cells and other components of the immune system. STAT3 signaling is important for mbIL21-mediated proliferation of human NK cells in maintaining NK cell proliferation and cytotoxicity. However, at the genome-wide level, the STAT3 transcriptome that modulates genes towards the ex vivo NK cell expansion has never been reported.

To this end, we performed STAT3 ChIP-seq and RNA-seq on resting human peripheral blood (naïve) and ex vivo expanded human NK cells, before and after stimulation with IL-21, to better understand the molecular events regulated by STAT3. This knowledge of the STAT3-mediated transcriptome controlling chemokine and cytokine expression, cellular proliferation, and migration is valuable for improving the activity of NK cell-based immunotherapy. Here, a combination of genome-wide analysis methods and computational data integration identify direct and indirect STAT3 targets before and after NK cell expansion.

a) Materials and Methods

### (1) PBMC Acquisition and NK Cell Preparation

Anonymized normal donor buffy coats were obtained from the Regional Red Cross Blood Center (Columbus, Ohio). Peripheral blood mononuclear cells (PBMCs) were purified by centrifugation over Ficoll-Paque from healthy donor buffy coat samples. Fresh NK cells were purified to ≥95% purity (CD3-CD16/56+) with RosetteSep Human NK Cell Enrichment Cocktail (STEMCELL Technologies, Vancouver, BC, Canada). In some instances, NK cells were further purified to <1% CD3+ after expansion prior to ChIP-seq and RNA-seq analysis.

### (2) NK Cell Ex Vivo Expansion

K562-based feeder cells (CSTX002) were produced by genetic modification of parental K562 to express CD137L and mbIL21. NK cells were expanded from PBMCs in vitro by weekly stimulation with the feeder cells in the presence of 100 IU/mL of rhIL-2. Cells were cultured in RPMI 1640 (Cellgro/Mediatech, Manassas, Va.) supplemented with 10% fetal bovine serum (HyClone, Logan, Utah), 2 mM 1-glutamine (Gibco/Invitrogen, Carlsbad, Calif.), and 1% penicillin/streptomycin (Cellgro/Mediatech) with or without cytokines, as indicated.

### (3) Reagents

Recombinant human IL-2 (Proleukin) was purchased from Novartis Vaccines and Diagnostics (East Hanover, N.J.) and IL-21 from PeproTech (Rocky Hill, N.J.).

### (4) Western Blot Analysis of STAT3 Phosphorylation

After stimulation with IL-21 (20 ng/ml), cells were lysed in RIPA lysis buffer with protease and phosphatase inhibitors, and centrifuged at 15,000×g at 4° C. for 15 min. Supernatants were boiled in SDS reducing sample buffer and subjected to SDS-polyacrylamide gel electrophoresis and then electrophoretically transferred onto polyvinylidene difluoride membranes. After blocking in TBST (20 mM Tris-HCl (pH 7.5), 150 mM NaCl, 0.1% Tween 20) containing 5% dry fat skim milk, the membranes were probed with primary antibodies overnight at 4° C. Antibodies used in this study were: anti-phospho-STAT3 (Tyr-705), anti-STAT3, and anti-β-actin from Cell Signaling Technology (Cambridge, Mass.). The membranes were extensively washed with TBST and incubated with horseradish peroxidase-conjugated anti-mouse or anti-rabbit antibody for 1 h at room temperature. The membranes were washed twice with TBST for 15 min. Blots were visualized using Amersham ECL western blotting detection reagent (GE Healthcare Bio-Sciences, Marlborough, Mass.), according to the manufacturer's instructions.

### (5) Chromatin Immunoprecipitation (ChIP) Assay

Naïve and expanded NK cells were rested overnight in media without cytokines, and then stimulated with IL-21 (20 ng/ml) for 30 min. NK cells were then fixed with 1% formaldehyde for 15 min and quenched with 0.125 M glycine. Chromatin was isolated by the addition of lysis buffer, followed by disruption with a Dounce homogenizer. Lysates were sonicated, and the DNA sheared to an average length of 300-500 bp. Genomic DNA (Input) was prepared by treating aliquots of chromatin with RNase, proteinase K and heat for de-crosslinking, followed by ethanol precipitation. Pellets were resuspended, and the resulting DNA was quantified on a NanoDrop spectrophotometer. Extrapolation to the original chromatin volume allowed quantitation of the total chromatin yield.

An aliquot of chromatin (10 μg) was precleared with protein A agarose beads (Invitrogen). Genomic DNA regions of interest were isolated using (2 μg of) antibody against STAT3 (Santa Cruz, sc-482, Lot C0816). Complexes were washed, eluted from the beads with SDS buffer, and subjected to RNase and proteinase K treatment. Crosslinks were reversed by incubation overnight at 65° C., and ChIP DNA was purified by phenol-chloroform extraction and ethanol precipitation.

### (6) ChIP Sequencing (Illumina)

Illumina sequencing libraries were prepared from the ChIP and Input DNAs by the standard consecutive enzymatic steps of end-polishing, dA-addition, and adaptor ligation. After a final PCR amplification step, the resulting DNA libraries were quantified and sequenced on Illumina's NextSeq 500 (75 nt reads, single end). Reads were aligned to the human genome (hg38) using the BWA algorithm (default settings). Duplicate reads were removed and only uniquely mapped reads (mapping quality ≥25) were used for further analysis. Alignments were extended in silico at their 3′-ends to a length of 200 bp, which is the average genomic fragment length in the size-selected library and assigned to 32-nt bins along the genome. The resulting histograms (genomic “signal maps”) were stored in bigWig files. Peak locations were determined using the MACS algorithm (v2.1.0) with a cutoff of p-value=le-7.

### (7) RNA-Seq Sample Preparation and Sequencing

Total RNA was purified from IL-21-stimulated naïve and expanded NK cells using the Total RNA Purification Plus Kit (Norgen Biotek, Ontario, Canada). The resulting total RNA was quantified in a Nanodrop ND-1000 spectrophotometer, checked for purity and integrity in a Bioanalyzer-2100 device (Agilent Technologies Inc., Santa Clara, Calif.) and submitted to the genomics core at the Nationwide Children's Hospital for sequencing. Libraries were prepared using the TruSeq RNA Sample Preparation Kit (Illumina Inc.) according to the protocols recommended by the manufacturer. Library quality was determined via Agilent 4200 Tapestation using a High Sensitivity D1000 ScreenTape Assay kit and quantified by KAPA qPCR (KAPA BioSystems). Approximately 60-80 million paired-end 150 bp sequence reads per library were generated using Illumina HiSeq4000 platform.

Sequencing reads from each sample were aligned to the GRCh38.p9 assembly of the Homo sapiens reference from NCBI using version 2.5.2b of the splice-aware aligner STAR. Feature coverage counts were calculated with HTSeq, using the GFF file that came with the assembly from NCBI. The default options for feature type, exon, and feature identifier, gene id, from the GFF were used to identify features for RNA-Seq analysis. Quality control checks for sample preparation and alignment were performed using custom Perl scripts which count types of reads using STAR's mapping quality metric and number of reads aligned to each feature class defined by the feature table that came with the assembly from NCBI. Differential expression analysis was performed using custom R scripts using DESeq2. Significantly differentially expressed features were identified with the criteria of a fold change of absolute value 1.5 and an adjusted p-value of ≤0.10 (10% FDR).

### (8) Integrative Omics Data Analyses

Identification of molecular network interactions and pathway analysis of differentially expressed genes at the RNA level were completed using the Ingenuity Pathway Analysis (Ingenuity Systems). The ingenuity knowledge base (genes only) with direct and indirect relationships was used and only molecules and/or relationships that had been experimentally observed in human were considered.

b) Results

### (1) Genome-Wide Identification of STAT3-Binding Sites in NK Cells

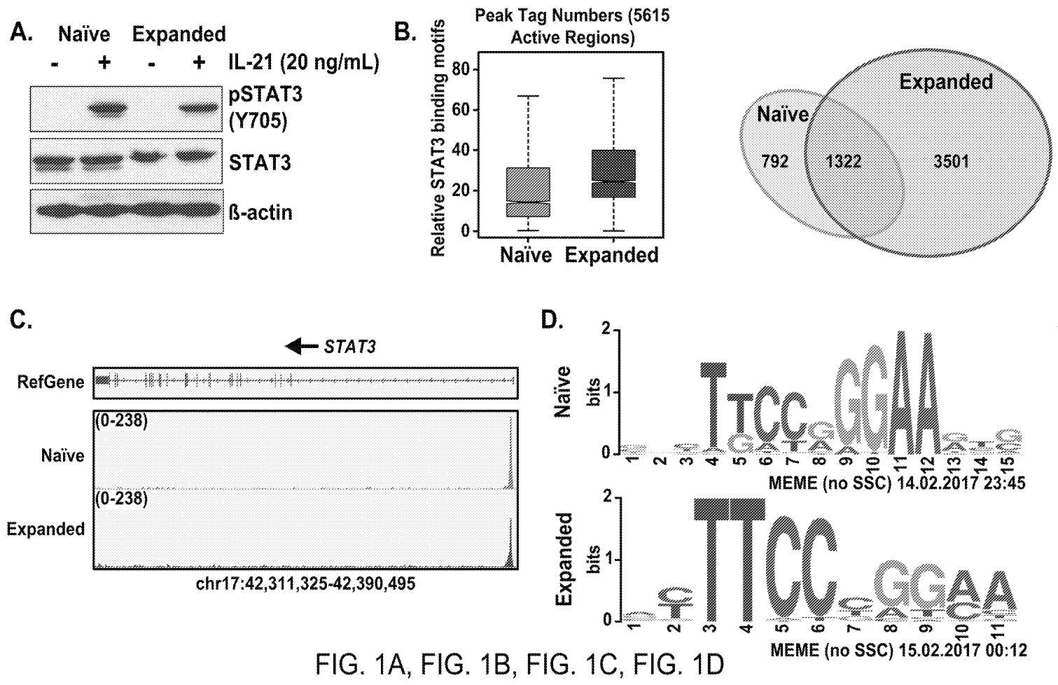

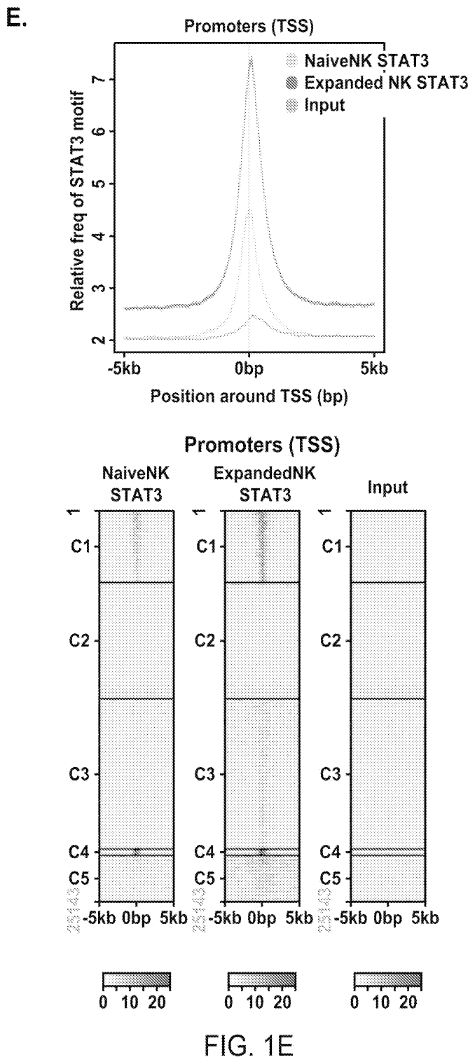

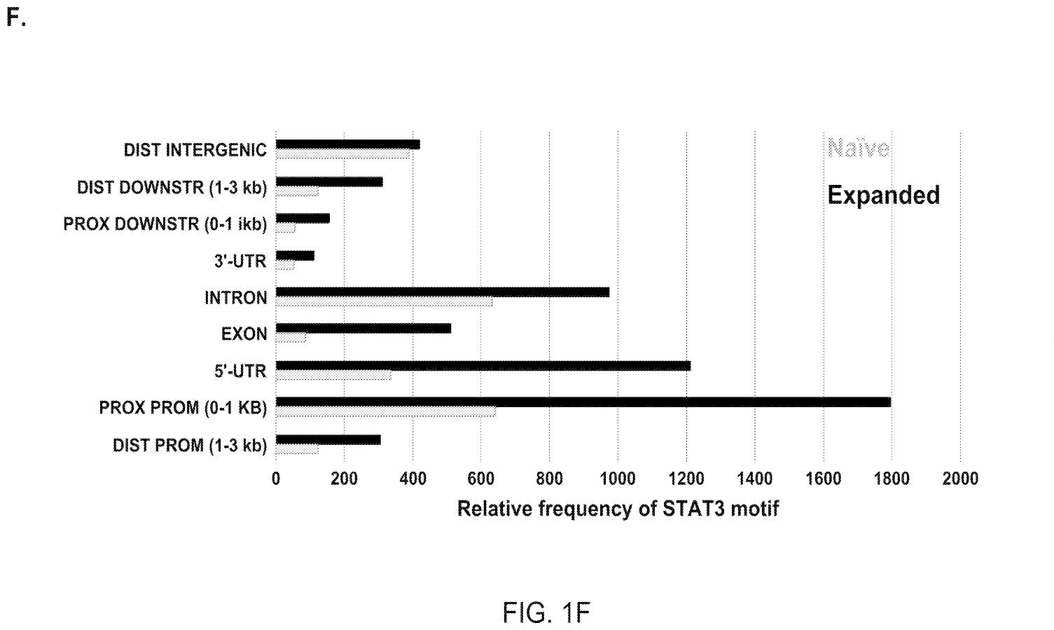

To characterize the regions bound by STAT3 in response to STAT3 phosphorylation by IL-21 in naïve and expanded NK cells in vivo, we sequenced STAT3-bound fragments by ChIP-seq. For this, primary NK cells were first treated with IL-21 (20 ng/mL) for 30 min and robust STAT3 phosphorylation was observed, whereas phosphorylated STAT3 was undetectable in the absence of IL-21 (FIG. 1A). These findings corroborate earlier findings that IL-21 induces strong and sustained STAT3 phosphorylation in NK cells. Next, chromatin immunoprecipitation of STAT3, both in IL-21-treated naïve and expanded cells, was coupled to high-throughput sequencing. The fragmented chromatin was immunoprecipitated and DNA deep-sequenced, as was non-immunoprecipitated total input DNA to generate the control ChIP-seq library. Peak identification was performed using model-based analysis for ChIP-seq (MACS) enrichment compared to the control libraries. Using a threshold p-value <10⁻⁷ and a <20% false discovery rate (FDR) in STAT3 immunoprecipitation versus input comparison, we identified 2,162 and 4,876 peaks bound by STAT3 in the IL-21-treated naïve and the expanded NK cells, respectively, and mapped them to the human genome with respect to transcriptional start sites (TSS). About 1800 were associated to a TSS (from −500 to +100 nt) in expanded NK cells and about 650 in naïve NK cells. The TSS-distal peaks were defined as intragenic, close intergenic (<20 Kb from TSS), and far intergenic (>20 Kb) (FIG. 1B). Overall, the data indicate that IL-21 stimulation in NK cells results in preferential binding of pSTAT3 to promoters.

STAT3 regulates its own transcription, a positive self-regulatory pattern distinctive of many transcription factors (TFs) that work to stabilize their own expression. We identified a clear peak at the promoter of the STAT3 gene itself in IL-21—stimulated naïve—and expanded-NK cells (FIG. 1C), indicating good quality of the STAT3 ChIP-seq library for identifying known STAT3-regulated genes. We scanned all STAT3-bound peaks for the de novo motif discovery using HOMER, and recovered the prototypical STAT3 homodimer motif TTCCnGGAA (FIG. 1D), although the dominant orientation was reversed in expanded NK cells compared to naive. For peaks within 5 kb of the promoter region we found higher STAT3 homodimer motif frequencies in the expanded NK cells than in naïve (FIG. 1E). Finally, with regards to the location of STAT3 motif we found that there was 3 to 5-fold increase in the STAT3 binding to the transcriptionally active regions (TARs) in expanded NK cells (FIG. 1F). There was no difference in the distal intergenic binding indicating that TAR binding was preferentially increased in the expanded NK cells. The analyses indicate that the list of STAT3-bound peaks is a high-quality dataset accurately reflecting active STAT3 gene regulation during the ex vivo expansion of human NK cells.

### (2) STAT3-Binding Sites Associated with Biological Processes

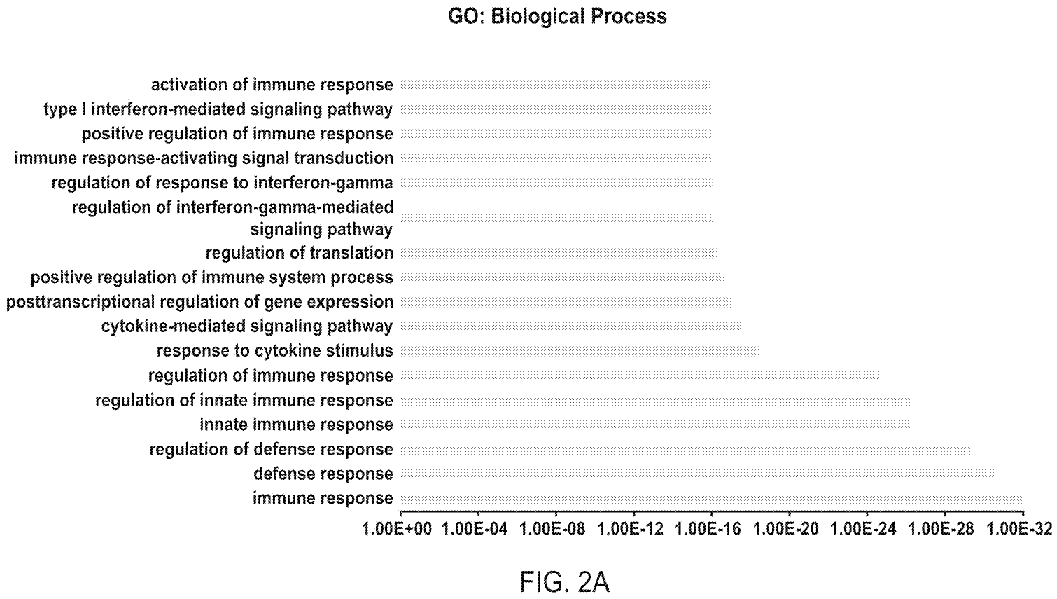

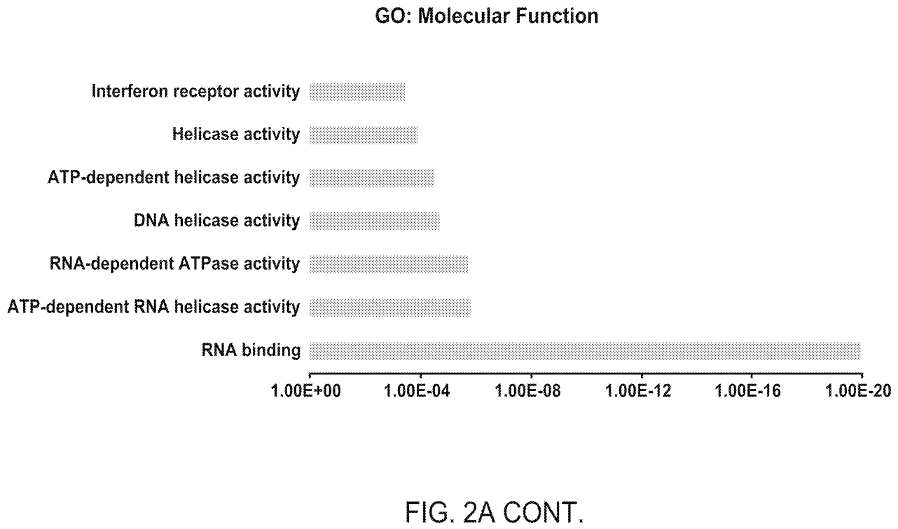

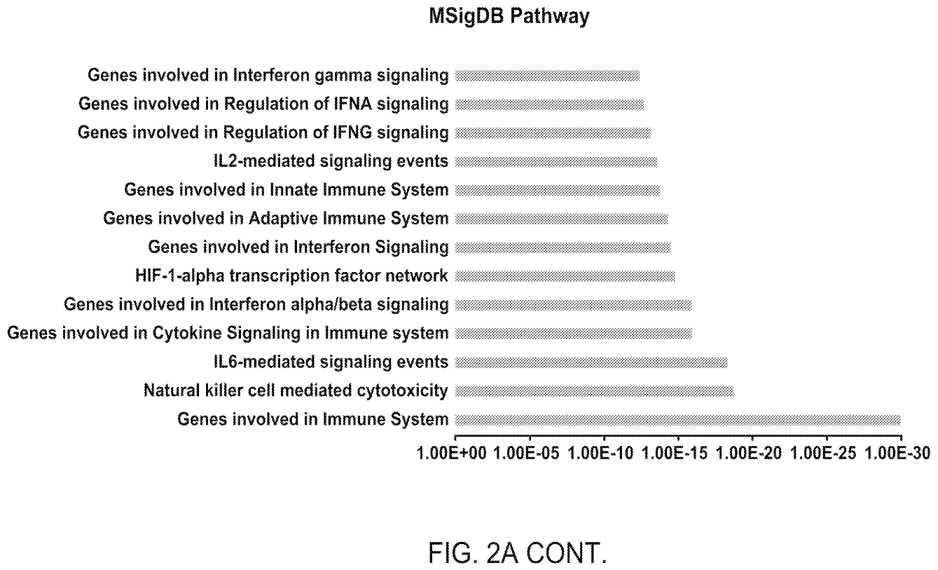

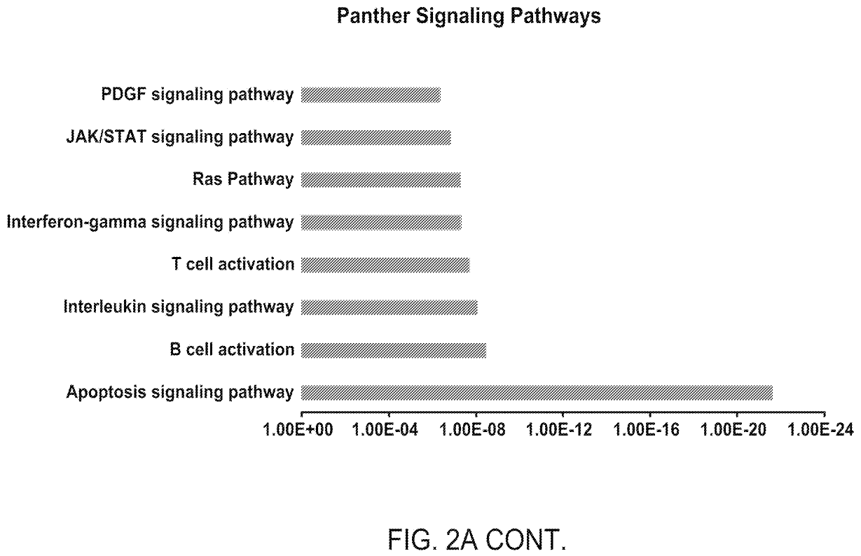

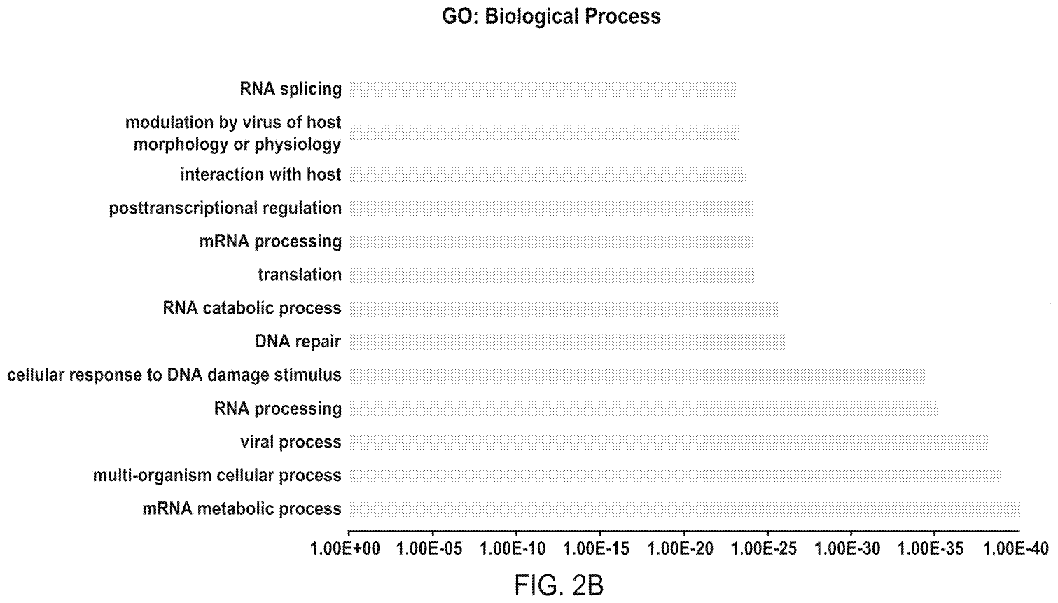

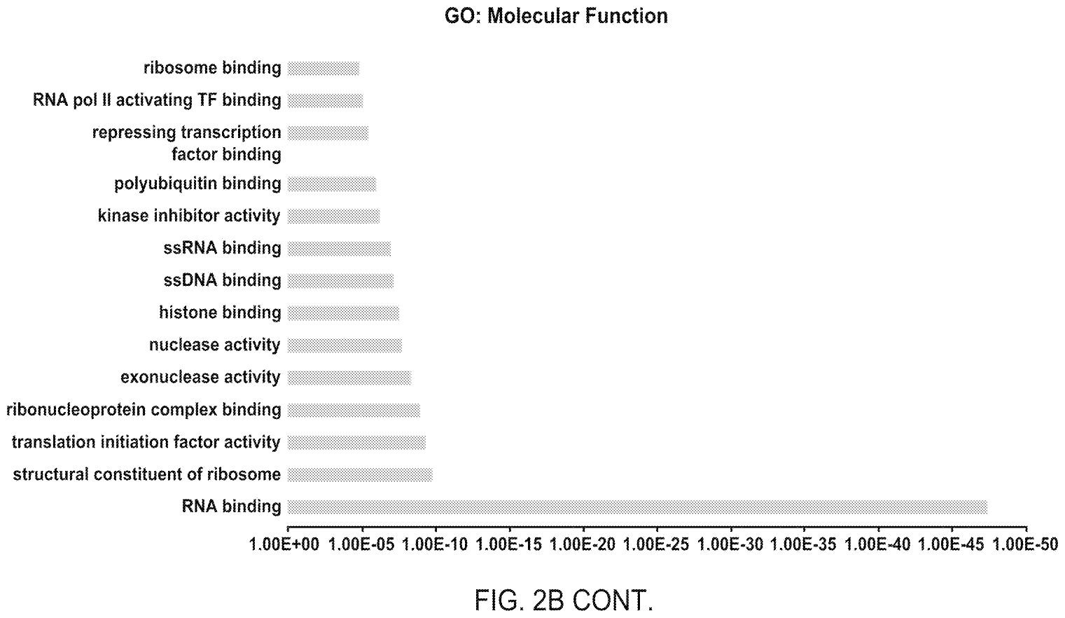

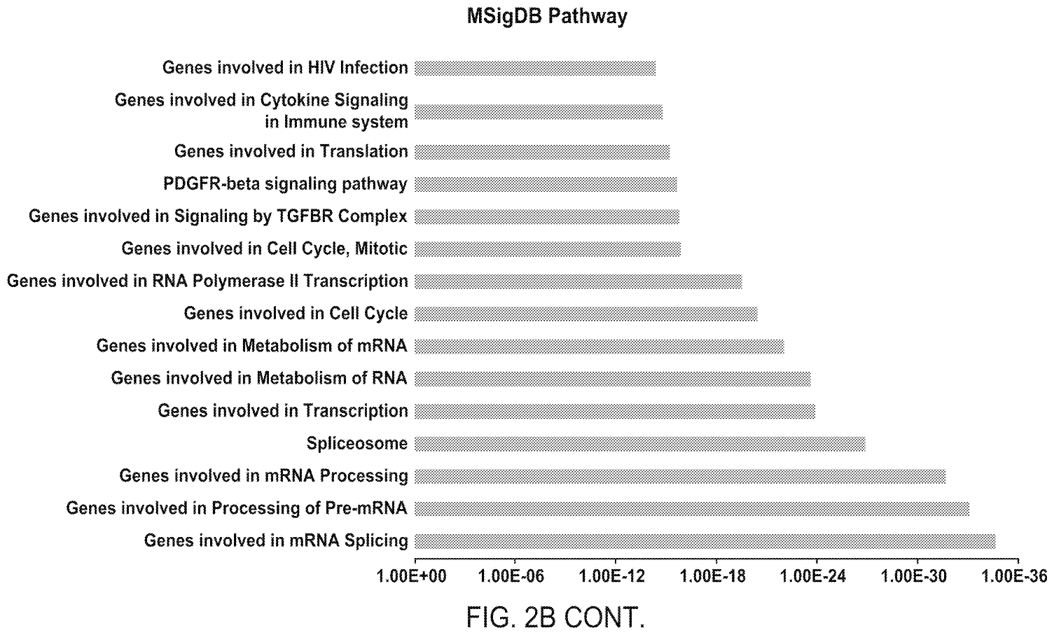

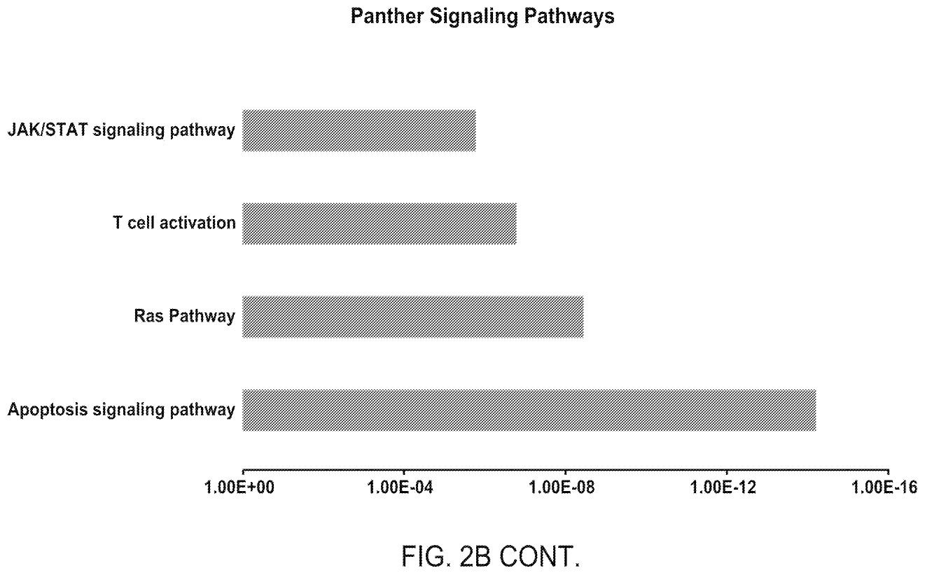

GREAT was used to interpret the genome-wide functional properties of the STAT3-binding sites. Whereas most tools that use Gene Ontology (GO) terms to derive functional annotations base their calculations on a set of genes or binding events that are proximal to genes (thus discarding most binding events), GREAT considers the nonrandom distribution of the genome and is specifically suited for the genome-wide analysis of ChIP-seq data. GREAT has been shown to outperform standard GO term-enrichment methods, and the analysis on the set of STAT3-binding sites reported a clear enrichment of key immune functions. FIG. 2 shows gene set enrichment values associated with various categories (GO Biological Process, GO Molecular Function, PANTHER Pathway, MSigDB Pathway). The top ontology on the Molecular Function enrichment was RNA-binding, -metabolism and -helicase activity both in naïve- and expanded-NK cells. In the MSigDB pathway category, multiple immune-related pathways, including “NK cell mediated cytotoxicity” and “cytokine signaling” were found to be enriched in naïve NK cells whereas mRNA processing, cell cycle, and cytokine signaling ontologies were clearly enriched in the expanded NK cells. Looking at the PANTHER pathway categories, terms related to apoptosis, interleukin signaling and JAK-STAT signaling were overexpressed indicating that the role of IL-21/JAK/STAT3 pathway in expanded NK cells is to modulate cell proliferation and survival, and to buttress its own signaling pathway. The GO Biological Process categories were enriched in terms related to immune response in naïve NK cells and to viral response and cellular proliferation in expanded NK cells. Altogether, the GREAT analysis provided an evidence indicating that the STAT3-binding events observed in naïve- and expanded NK cells regulate immune cell functions and cell growth.

### (3) Differential Gene Expression in Naïve and Expanded NK Cell

In an effort to define unique and common signatures of NK cell activity in response to IL-21 stimulation, we studied the entire transcriptome of NK cells. High-throughput RNA sequencing (RNA-seq) was used to investigate expression profiles of resting and IL-21-stimulated naïve and expanded NK cells in five donors (FIG. 3). Approximately one billion bp were sequenced, consisting of ˜60-80 million paired-end 150 bp sequence reads for each sample. Sequencing reads from each sample were aligned to the GRCh38.p9 assembly of the Homo sapiens reference from NCBI using version 2.5.2b of the splice-aware aligner STAR. Approximately 96% of the reads were successfully aligned to the human genome. After combining the RNA-seq libraries from naïve- and expanded-NK cells, we were able to unambiguously assemble 79% and 78% of the transcripts, respectively.

We analyzed the transcriptome of the naïve and expanded NK cells by RNA-seq. We detected 14,183 RNA transcripts from the sense strand, and from these we considered only those RNAs whose level of expression (FPKM) was >1, in order to avoid false differential expression. We then set an arbitrary threshold to define STAT3-modulated genes, those in which activation of STAT3 resulted in an over 50% increase or decrease (fold change ratio >1.5) in expression. Approximately 7,951 transcripts were differentially expressed in expanded NK cells compared to naïve cells (FIG. 3A).

We found 2,938 overexpressed genes in which the expanded/naïve FPKM expression ratio was >1.5 and 5,013 down-regulated genes in which the fold change ratio was

<1.5 (FIG. 3B, Top 100 differently expressed genes shown in FIG. 3C). Thus, the most frequent effect of STAT3 stimulation in expanded NK cells was a down-regulation of gene expression. To examine biological pathways these differentially expressed transcripts are involved in, we next performed a gene set enrichment analysis (Enrichr) using the GO gene set to identify associations within each subset. Among the down-regulated genes in the expanded vs naïve NK cells after stimulation with IL-21, GO analysis showed several statistically significant categories, mainly “transcription regulation”. On the other hand, the genes upregulated in expanded NK cells were more significantly enriched in cell growth category such as “DNA metabolic processes”, “regulation of mitosis”, “mitochondrial ATP synthesis”, “glycolysis” and “p53 pathway” (FIG. 3D).

We used the IPA package (QIAGEN Redwood City) to identify pathways to which differential expressed genes (DEGs) belong, as well as to explore the existence of signaling networks connecting these DEGs. Several pathways were significantly enriched in the dataset of 4,275 DEGs (FIG. 4A). Amongst the most enriched pathways were oxidative phosphorylation, nucleotide excision repair (NER) pathway, sirtuin signaling pathway, cell cycle, and Granzyme A signaling. Differentially expressed mRNA genes were also grouped in gene regulatory networks with the IPA software. We found 25 regulatory networks related with a variety of functions, and the top-scoring ones were that of Cell Death and Survival, Infectious Diseases, Cellular Function and Maintenance, and Hematological Disease (FIG. 4B).

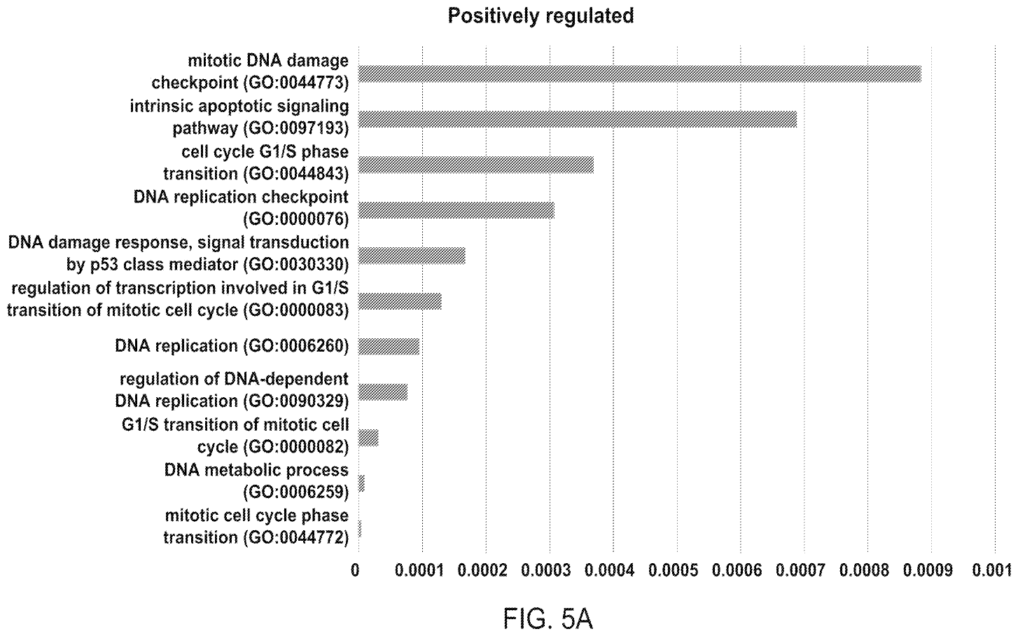

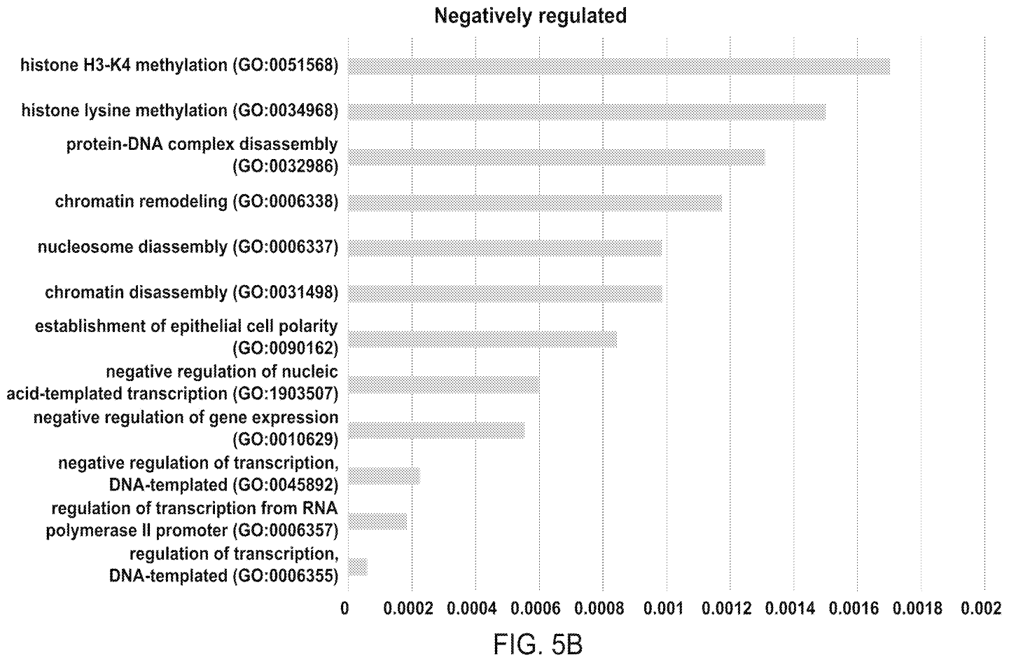

We then integrated the STAT3's genome-wide binding pattern with expression data (RNA-seq) to unravel the major effect STAT3 has on the genes it regulates. STAT3-binding sites were annotated to the nearest expressed gene (TSS) according to the RNA-seq data and binned on the distance to the closest TSS. Nearly ˜50% of the STAT3-binding sites preferentially lie within gene bodies, and only ˜10% are found at distances greater than 200 kb from the nearest TSS. To obtain a global view of the changes in gene expression on IL-21 stimulation, after the STAT3 binding regions with significant binding differences were identified, we associated them with nearby genes and then the differentially-regulated transcripts were examined for over-represented GO terms using Enrichr (FIG. 5). Genes with GO Biological Processes categories related to cell division, apoptosis, and DNA damage response were enriched in expanded NK cells relative to naïve cells (FIG. 5A). Whereas, GO terms related with chromatin remodeling, nucleosome disassembly, and negative regulation of transcription was enriched in the naïve cells (FIG. 5B). The expression of these pathways prime NK cells to expand rapidly and explains why IL-21 stimuli is so potent than their fresh counterparts.

c) Discussion

NK cells are innate lymphocytes with enormous phenotypic and functional diversity. They are major players of the innate immune system and immediate effector cells against viral infections, pathogens, and tumor cells; making them a promising tool for the use in adoptive immunotherapy. However, NK-cells compromise only 5-15% of circulating blood lymphocytes; thus, the major obstacle for adoptive NK cell immunotherapy is obtaining sufficient cell numbers. In this context, ex vivo cultivation is an attractive option to increase NK cells in numbers and to improve their antitumor potential prior to clinical applications. Earlier, we have shown that STAT3 is a key signal transducer that regulates gene expression in these expanded NK cells. Binding of IL-21 to IL-21R on NK cells activates STAT3; IL-21 induced phosphorylation of STAT3 in expanded NK cells with a maximal response after 30 min and return to baseline by 6 hrs.

In an effort to uncover the identities of the STAT3-regulated genes that modulate the NK cell activity after expansion, here we report the first genome-wide map of STAT3-binding sites both in human naïve and ex vivo expanded NK cells, on IL-21 stimulation. ChIP-seq is the most powerful and direct means to locate genes controlled by a transcription factor in vivo, offering better coverage, higher resolution, and less noise. Most STAT3-binding sites preferentially locate within gene bodies or in adjacent regions, and STAT3 binding usually occurs in evolutionary conserved genomic regions. We identified 3,501 high-quality STAT3-binding sites specifically in expanded NK cells on IL-21 stimulation, 792 were also identified in naïve cells and 1322 binding regions were similar in both, which indicates that phosphorylated STAT3 exerts a very specific transcriptional response during the expansion. Furthermore, GREAT analysis provided strong evidence for the involvement of the STAT3-binding sites in cell growth and immune functions (e.g., “interleukin signaling,” “JAK-STAT signaling pathways,” NK cell mediated cytotoxicity, cytokine signaling, and apoptosis). These observations support the earlier report that STAT3 has an immune-activating role in NK cells, and STAT3 phosphorylation regulates NK cell proliferation, and cytotoxicity.

After correcting for multiple testing, 6,464 genes, displaying a wide array of functional roles, showed a significant differentially expression between expanded and naïve NK cells. For instance, one of the highest expressed gene in the expanded NK cells was BIRC5 (Survivin) which is a member of the inhibitor of apoptosis (IAP) gene family, encoding negative regulatory proteins that prevent apoptotic cell death. Interestingly, BIRC5, is essential for the development of NK cells and high expression of BIRC5 in NK cell populations has been observed in the bone marrow and were consistent with a non-redundant role for BIRC5 in innate lymphocyte development. In mice with cell-specific deletions of the BIRC5 gene steady-state populations of NK cells were severely reduced. The findings highlight and support crucial role of BIRC5 in the proliferation and maturation of NK cells. Further, several promotors for cell survival and proliferation including MK167 (Ki-67), TOP2A, CKS2, and RACGAP1 were upregulated by IL-21 whereas antiproliferative or proapoptotic proteins such as PTCH1, TGFB3, and ATM were downregulated. Thus, gene expression signatures are consistent with improved functional activity of NK cells after cytokine stimulation.

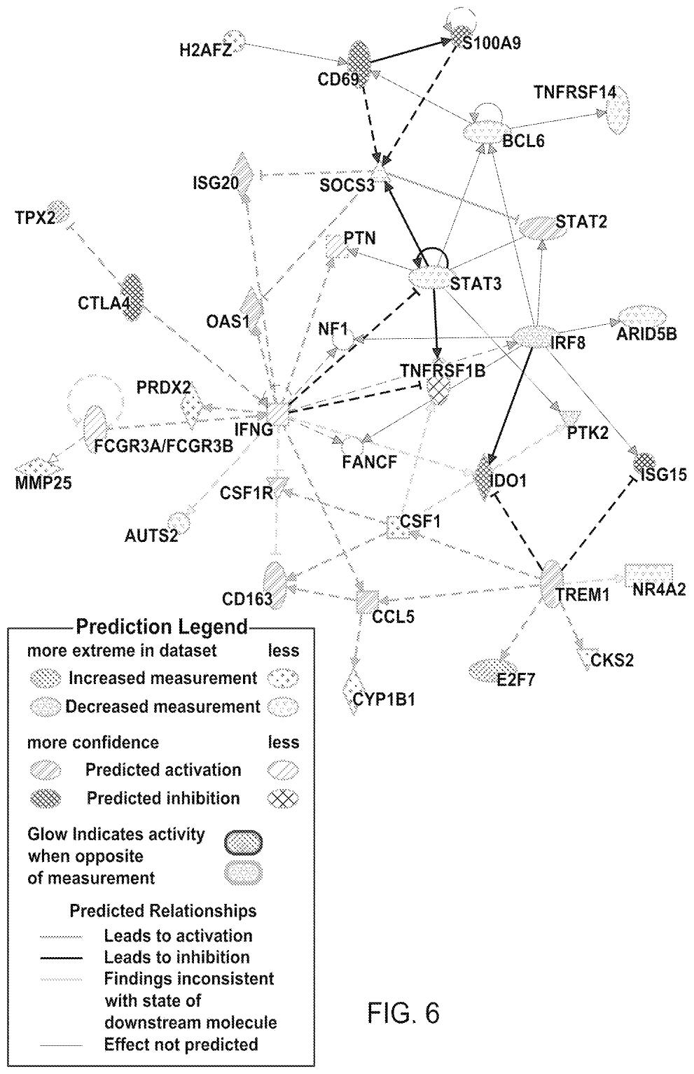

The combination of whole-genome profiling approaches shows differential STAT3 binding sites near almost a 20% of all genes that differ in expression between naïve and expanded NK cells, indicating that STAT3 is a regulator of the robust growth and cytotoxicity displayed by the expanded NK cell subtype. The STAT3 binding and expression results indicate possible models by which STAT3 regulates biological pathways to promote growth and maturation in NK cells. Ontological analysis of the genes up-regulated in expanded NK cells with increased nearby STAT3 binding indicates that STAT3 promotes cell proliferation, higher glycolytic metabolism, division, and the immune response (FIG. 5A). Furthermore, it ensures continued high expression of IFNγ by up-regulating its pathway partners and upstream signaling molecules and promoting cross-talk with the colony stimulating factor pathway (FIG. 6). Most critically, STAT3 strongly up-regulates many mechanisms of apoptosis resistance. These effects synergize and likely lead to increased cell proliferation and activation of NK cells during an ex vivo expansion.

Such novel insights into the genetics, epigenetics, transcriptome and proteome of expansion of NK cells and understanding of IL-21's impact on human NK cells bolster implementation of ex vivo expanded NK cells in adoptive NK-cell therapy. Furthermore, these results provide data to identify a substantial number of plausible target molecules for the generating more effective modified NK cells, for example with CARs or other modifications, for treatment-resistant malignancies.

### 2. Example 2: Differentially Regulated Genes are Epigenetically Regulated or Epigenetic Regulators

We took the top 200 differentially-expressed genes from the RNAseq naïve-vs-expanded comparison, and then searched for those genes in a separate list of differentially-expressed genes from the ATACseq naïve-vs-expanded comparison. 38 of the RNAseq genes were also in the ATACseq list. Thus, 19% of the differentially-expressed genes in expanded NK cells are affected by epigenetic changes.

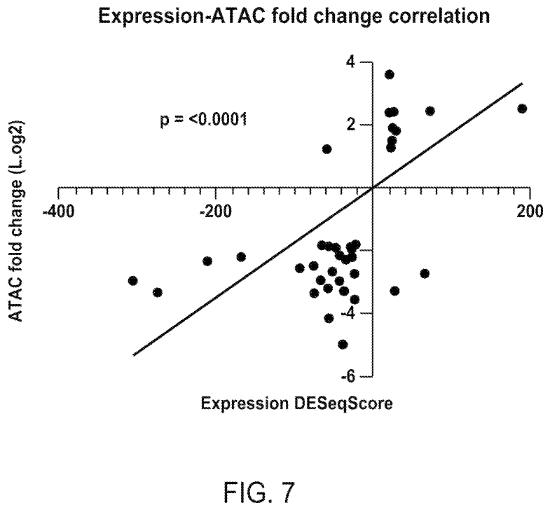

We then plotted the DESeqScore of the RNAseq with the log 2 fold-change from the ATACseq, to see if there was a direct correlation with opening of the chromatin and gene expression. All but 3 of the 38 genes correlated in the expected direction, with high statistical significance (FIG. 7). Therefore, epigenetic regulation of chromatin accessibility is highly correlated with modulation of gene expression in expanded NK cells.

Among the 200 top differentially-expressed genes, 27 of them are histones, and only one of those are among the other 38 genes described above. This means that ⅓ (65) of the top 200 differentially-regulated genes in expanded NK cells are either epigenetic regulators or are epigenetically regulated. Those genes are: HIST1H1B, HIST1H2AB, HIST1H2AG, HIST1H2AH, HIST1H2AI, HIST1H2AJ, HIST1H2AL, HIST1H2BB, HIST1H2BE, HIST1H2BH, HIST1H2BJ, HIST1H2BL, HIST1H2BM, HIST1H2BO, HIST1H3B, HIST1H3C, HIST1H3F, HIST1H3G, HIST1H3H, HIST1H3J, HIST1H4A, HIST1H4D, HIST1H4L, HIST2H3C, HIST2H4B, HIST3H2BA, HIST3H2BB, ABCA1, AK5, ASCL2, B3GAT1, CACNA2D2, CCR5, CXCR2, ENPP5, FBLN2, FCRL3, GNAL, GPRASP1, GRIK4, GTSE1, HIST3H2BB, IL1B, ITGA2, KIF13A, KIF4A, LINC00599, LONRF3, LRRN3, MSC-AS1, NFIX, NUAK1, NUAK2, PCDH1, PLXNA4, RAD51, RNF157, SLC1A7, SPON2, TYMS, WWC2, ZNF442, ZNF727, and ZSCAN18.

### E. REFERENCES

Beaulieu, A., and Sun, J. (2015). An essential role for Birc5 in innate lymphocyte development (HEM5P.223). The Journal of Immunology 194, 120.123-120.123.
Cacalano, N. A. (2016). Regulation of Natural Killer Cell Function by STAT3. Front Immunol 7, 128.
Chen, E. Y., Tan, C. M., Kou, Y., Duan, Q., Wang, Z., Meirelles, G. V., Clark, N. R., and Ma'ayan, A. (2013). Enrichr: interactive and collaborative HTML5 gene list enrichment analysis tool. BMC Bioinformatics 14, 128.
Denman, C. J., Senyukov, V. V., Somanchi, S. S., Phatarpekar, P. V., Kopp, L. M., Johnson, J. L., Singh, H., Hurton, L., Maiti, S. N., Huls, M. H., Champlin, R. E., Cooper, L. J., and Lee, D. A. (2012). Membrane-bound IL-21 promotes sustained ex vivo proliferation of human natural killer cells. PLoS One 7, e30264.
Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21.
Fang, F., Xiao, W., and Tian, Z. (2017). NK cell-based immunotherapy for cancer. Semin Immunol 31, 37-54.
Fehniger, T. A., Cooper, M. A., Nuovo, G. J., Cella, M., Facchetti, F., Colonna, M., and Caligiuri, M. A. (2003). CD56bright natural killer cells are present in human lymph nodes and are activated by T cell-derived IL-2: a potential new link between adaptive and innate immunity. Blood 101, 3052-3057.
Gao, X., Mi, Y., Guo, N., Xu, H., Xu, L., Gou, X., and Jin, W. (2017). Cytokine-Induced Killer Cells As Pharmacological Tools for Cancer Immunotherapy. Front Immunol 8, 774.
Gotthardt, D., and Sexl, V. (2016). STATs in NK-Cells: The Good, the Bad, and the Ugly. Front Immunol 7, 694.
Granzin, M., Wagner, J., Kohl, U., Cerwenka, A., Huppert, V., and Ullrich, E. (2017). Shaping of Natural Killer Cell Antitumor Activity by Ex Vivo Cultivation. Front Immunol 8, 458.
Habib, T., Nelson, A., and Kaushansky, K. (2003). IL-21: a novel IL-2-family lymphokine that modulates B, T, and natural killer cell responses. J Allergy Clin Immunol 112, 1033-1045.
Hosseini, E., Ghasemzadeh, M., Kamalizad, M., and Schwarer, A. P. (2017). Ex vivo expansion of CD3 (depleted) cord blood-MNCs in the presence of bone marrow stromal cells; an appropriate strategy to provide functional NK cells applicable for cellular therapy. Stem Cell Res 19, 148-155.
Ishikawa, T., Okayama, T., Sakamoto, N., Ideno, M., Oka, K., Enoki, T., Mineno, J., Yoshida, N., Katada, K., Kamada, K., Uchiyama, K., Handa, O., Takagi, T., Konishi, H., Kokura, S., Uno, K., Naito, Y., and Itoh, Y. (2018). Phase I clinical trial of adoptive transfer of expanded natural killer cells in combination with IgGI antibody in patients with gastric or colorectal cancer. Int J Cancer.
Kuleshov, M. V., Jones, M. R., Rouillard, A. D., Fernandez, N. F., Duan, Q., Wang, Z., Koplev, S., Jenkins, S. L., Jagodnik, K. M., Lachmann, A., Mcdermott, M. G., Monteiro, C. D., Gundersen, G. W., and Ma'ayan, A. (2016). Enrichr: a comprehensive gene set enrichment analysis web server 2016 update. Nucleic Acids Res 44, W90-97.
Kwon, H., Thierry-Mieg, D., Thierry-Mieg, J., Kim, H. P., Oh, J., Tunyaplin, C., Carotta, S., Donovan, C. E., Goldman, M. L., Tailor, P., Ozato, K., Levy, D. E., Nutt, S. L., Calame, K., and Leonard, W. J. (2009). Analysis of interleukin-21-induced Prdm1 gene regulation reveals functional cooperation of STAT3 and IRF4 transcription factors. Immunity 31, 941-952.
Li, H., and Durbin, R. (2009). Fast and accurate short read alignment with Burrows-Wheeler transform. Bioinformatics 25, 1754-1760.
Li, Q., Ye, L. J., Ren, H. L., Huyan, T., Li, J., Shi, J. L., and Huang, Q. S. (2015). Multiple effects of IL-21 on human NK cells in ex vivo expansion. Immunobiology 220, 876-888.
Love, M. I., Huber, W., and Anders, S. (2014). Moderated estimation of fold change and dispersion for RNA-seq data with DESeq2. Genome Biol 15, 550.
Mclean, C. Y., Bristor, D., Hiller, M., Clarke, S. L., Schaar, B. T., Lowe, C. B., Wenger, A. M., and Bejerano, G. (2010). GREAT improves functional interpretation of cis-regulatory regions. Nat Biotechnol 28, 495-501.
Ni, L., Wang, L., Yao, C., Ni, Z., Liu, F., Gong, C., Zhu, X., Yan, X., Watowich, S. S., Lee, D. A., and Zhu, S. (2017). The histone deacetylase inhibitor valproic acid inhibits NKG2D expression in natural killer cells through suppression of STAT3 and HDAC3. Sci Rep 7, 45266.
Preethy, S., Dedeepiya, V. D., Senthilkumar, R., Rajmohan, M., Karthick, R., Terunuma, H., and Abraham, S. J. K. (2017). Natural killer cells as a promising tool to tackle cancer-A review of sources, methodologies, and potentials. Int Rev Immunol 36, 220-232.
Rajbhandary, S., Zhao, M. F., Zhao, N., Lu, W. Y., Zhu, H. B., Xiao, X., Deng, Q., and Li, Y. M. (2013). Multiple cytotoxic factors involved in IL-21 enhanced antitumor function of CIK cells signaled through STAT-3 and STATSb pathways. Asian Pac J Cancer Prev 14, 5825-5831.
Somanchi, S. S., and Lee, D. A. (2016). Ex Vivo Expansion of Human NK Cells Using K562 Engineered to Express Membrane Bound IL21. Methods Mol Biol 1441, 175-193.
Spolski, R., and Leonard, W. J. (2008). Interleukin-21: basic biology and implications for cancer and autoimmunity. Annu Rev Immunol 26, 57-79.
Vivier, E., Tomasello, E., Baratin, M., Walzer, T., and Ugolini, S. (2008). Functions of natural killer cells. Nat Immunol 9, 503-510.
Wang, X., Lee, D. A., Wang, Y., Wang, L., Yao, Y., Lin, Z., Cheng, J., and Zhu, S. (2013). Membrane-bound interleukin-21 and CD137 ligand induce functional human natural killer cells from peripheral blood mononuclear cells through STAT-3 activation. Clin Exp Immunol 172, 104-112.
Zhang, Y., Liu, T., Meyer, C. A., Eeckhoute, J., Johnson, D. S., Bernstein, B. E., Nusbaum, C., Myers, R. M., Brown, M., Li, W., and Liu, X. S. (2008). Model-based analysis of ChIP-Seq (MACS). Genome Biol 9, R137.
Zhu, S., Phatarpekar, P. V., Denman, C. J., Senyukov, V. V., Somanchi, S. S., Nguyen-Jackson, H. T., Mace, E. M., Freeman, A. F., Watowich, S. S., Orange, J. S., Holland, S. M., and Lee, D. A. (2014). Transcription of the activating receptor NKG2D in natural killer cells is regulated by STAT3 tyrosine phosphorylation. Blood 124, 403-411.

1. An expanded modified natural killer cell comprising a modified STAT3 transcriptome.

2. The expanded modified natural killer cell of claim 1, comprising one or more differentially expressed genes, wherein a differentially expressed gene has at least about 50% increased or decreased expression relative to expression of the gene in a naïve natural killer cell.

3. The expanded modified natural killer cell of claim 1, wherein the STAT3 transcriptome comprises one or more differentially expressed genes involved in telomere organization.

4. The expanded modified natural killer cell of claim 1, wherein the STAT3 transcriptome comprises differentially expressed genes involved in the regulation of mitosis.

5. The expanded modified natural killer cell of claim 1, wherein the STAT3 transcriptome comprises differentially expressed genes involved in DNA repair.

6. The expanded modified natural killer cell of claim 1, wherein the STAT3 transcriptome comprises differentially expressed genes involved in immunity.

7. The expanded modified natural killer cell of claim 1, wherein the STAT3 transcriptome comprises differentially expressed genes involved in cytokine signaling.

8. The expanded modified natural killer cell of claim 1, wherein the STAT3 transcriptome comprises differentially expressed genes involved in glycolysis.

9. The expanded modified natural killer cell of claim 1, wherein the STAT3 transcriptome comprises differentially expressed genes involved in gluconeogenesis.

10. The expanded modified natural killer cell of claim 1, wherein the STAT3 transcriptome comprises differentially expressed genes involved in the p53 pathway.

11. The expanded modified natural killer cell of claim 1, wherein the STAT3 transcriptome comprises one or more differentially expressed genes comprising SPON2, FCRL3, KIF13A, PDZD4, PLXNA4, HIST2H3A, LINC00599, RPL38P4, LRRN3, ITGA2, HIST2H3C, IQGAP3, MSC, DAB2, ENPP5, LYZ, PCDH1, NCKAP5L, PTK2, RIMKLB, GPRASP1, PTGDS, DTX1, FBLN2, COL6A1, MGAM, ZNF727, TGFA, LOC105373204, ZNF135, ZNF835, SLC1A7, LINC00565, LRRN1, DGKK, VIPR2, GRIK4, ZMAT4, LOC105369772, ZNF667, IL1B, MSRB3, TCF7L2, PODN, LINC00469, GNAIl, LOC105369723, HIST1H3G, HIST1H2AJ, TOP2A, HIST1H3C, HIST1H2AB, HISTiHiB, HIST1H3B, HIST1H3F, ASPM, HIST1H2AL, MKI67, RRM2, HIST1H3J, CCNA2, CDK1, TPX2, NCAPG, KIF2C, CENPA, PBK, HIST3H2BA, E2F8, CDC25C, FOXM1, KIF18B, APOBEC3B, PLK1, CDKN3, SAPCD2, DLGAP5, BIRC5, CEP55, HIST1H2BM, KIF14, TTK, ANLN, CDCA2, BUB1B, KIF23, NEK2, KIF20A, CDC20, CCNB2, HJURP, UBE2C, SPC24, AURKB, DEPDC1, KIF4A, MYBL2, MELK, CKAP2L, SHCBP1, LIG1, MTBP, CDC1, TIMM23, RAD9A, CTSZ, HMGB2, JAG1, PDCD1LG2, SH2B3, UXT, CHEK1, CD274, CD86, HMGA1, STAT3, IRF1, STAT2, IRS, SLAMF1, FGFBP2, MKI67, CTLA4, ABL1, IRF8, IL7R, PROK2, FAR2, PRKD3, MCM7, BCL6, IRF4, JAK1, HIF1A, C1QBP, JAKMIP1, RBPJ, KIF15, RAD51, TXNIP, KPNA2, TXN, ATM, IRF9, RAD21, CIITA, CD74, HLADRB1, RAD54B, CHEK2, CTCF, RUNX3, HLADRA, CADM1, BMI1, HK2, CD63, CD82, BNIP3, GLIPRI, or DLC1.

12. The expanded modified natural killer cell of claim 1, wherein the STAT3 transcriptome comprises differentially expressed one or more genes comprising HISTiHiB, HIST1H2AB, HIST1H2AG, HIST1H2AH, HIST1H2AI, HIST1H2AJ, HIST1H2AL, HIST1H2BB, HIST1H2BE, HIST1H2BH, HIST1H2BJ, HIST1H2BL, HIST1H2BM, HIST1H2BO, HIST1H3B, HIST1H3C, HIST1H3F, HIST1H3G, HIST1H3H, HIST1H3J, HIST1H4A, HIST1H4D, HIST1H4L, HIST2H3C, HIST2H4B, HIST3H2BA, HIST3H2BB, ABCA1, AK5, ASCL2, B3GAT1, CACNA2D2, CCR5, CXCR2, ENPP5, FBLN2, FCRL3, GNAL, GPRASP1, GRIK4, GTSE1, HIST3H2BB, IL1B, ITGA2, KIF13A, KIF4A, LINC00599, LONRF3, LRRN3, MSC-AS1, NFIX, NUAK1, NUAK2, PCDH1, PLXNA4, RAD51, RNF157, SLC1A7, SPON2, TYMS, WWC2, ZNF442, ZNF727, or ZSCAN18.

13. The expanded modified natural killer cell of claim 1, wherein the natural killer cell comprises an upregulated one or more proteins selected from the group consisting of BIRCS, MK167, TOP2A, CKS2 and RACGAP1.

14. The expanded modified natural killer cell of claim 1, wherein the natural killer cell comprises a downregulated one or more proteins selected from the group consisting of PTCH1, TGFB3, and ATM.

15. The expanded modified natural killer cells of claim 1, wherein the cells are expanded ex vivo by contacting the NK cell with a plasma membrane vesicle, an exosome, or a feeder cell that was engineered to express one or more of membrane bound IL-21, IL-15, and 4-BBL.

16. The expanded modified natural killer cells of claim 1, wherein the cells are expanded ex vivo by contacting the NK cell with one or more of IL-21, IL-15, aid/0 and 4-BBL.

17. The expanded modified natural killer cells of claim 1, wherein the cells are expanded in vivo by contacting the NK cell with a plasma membrane vesicle, an exosome, or a feeder cell that was engineered to express one or more of membrane bound IL-21, IL-15, and 4-BBL.

18. The expanded modified natural killer cells of claim 1, wherein the cells are expanded in vivo by contacting the NK cell with one or more of IL-21, IL-15, and 4-BBL.

19. A method of treating a cancer in a subject comprising administering to the subject a therapeutically effective amount of the expanded modified natural killer cells of claim 1.

20. A method of modulating the immune system of a subject, comprising administering an effective amount of expanded modified natural killer cells including an activated STAT3 transcriptome.
